SEQUENCE LISTING (42 sequences)

SEQ ID NO: 1 - protein, 121 aa, Synthetic construct
QVQLVQSGTEVRKPGSSVKVSCKTSGVTFNNYAITWVRQAPGQGLEWMGGIIPVFSPPNYAQKFQGRVTITADESTNTAYMELNSLRSDDTAIYFCAREGERGYTYGYDYWGQGTLVTVSS

SEQ ID NO: 2 - protein, 8 aa, Synthetic construct
GVTFNNYA

SEQ ID NO: 3 - protein, 8 aa, Synthetic construct
IIPVFSPP

SEQ ID NO: 4 - protein, 14 aa, Synthetic construct
AREGERGYTYGYDY

SEQ ID NO: 5 - protein, 108 aa, Synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 6 - protein, 6 aa, Synthetic construct
QSISSY

SEQ ID NO: 7 - skipped

SEQ ID NO: 8 - protein, 10 aa, Synthetic construct
QQSYSTPPIT

SEQ ID NO: 9 - protein, 447 aa, Synthetic construct
QVQLVQSGTEVRKPGSSVKVSCKTSGVTFNNYAITWVRQAPGQGLEWMGGIIPVFSPPNYAQKFQGRVTITADESTNTAYMELNSLRSDDTAIYFCAREGERGYTYGYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 215 aa, Synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 117 aa, Synthetic construct [sequence omitted]

SEQ ID NO: 12 - protein, 8 aa, Synthetic construct
GFTFSNFG

SEQ ID NO: 13 - protein, 8 aa, Synthetic construct
ISGGGRDT

SEQ ID NO: 14 - protein, 10 aa, Synthetic construct
VKWGNIYFDY

SEQ ID NO: 15 - protein, 107 aa, Synthetic construct [sequence omitted]

SEQ ID NO: 16 - protein, 6 aa, Synthetic construct
LSINTF

SEQ ID NO: 17 - protein, 9 aa, Synthetic construct
QQSSNTPFT

SEQ ID NO: 18 - protein, 443 aa, Synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 214 aa, Synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 121 aa, Synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSS

SEQ ID NO: 21 - protein, 8 aa, Synthetic construct
GVTFRNFA

SEQ ID NO: 22 - protein, 8 aa, Synthetic construct
IIPFFSAA

SEQ ID NO: 23 - protein, 14 aa, Synthetic construct
AREGERGHTYGFDY

SEQ ID NO: 24 - protein, 447 aa, Synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 215 aa, Synthetic construct; identical to SEQ ID NO: 10

SEQ ID NO: 26 - protein, 326 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 323 aa, Synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 28 - protein, 165 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 29 - protein, 133 aa, Homo sapiens [sequence omitted]

SEQ ID NO: 30 - protein, 15 aa, Synthetic construct
GGGGSGGGGSGGGGS

SEQ ID NO: 31 - protein, 25 aa, Synthetic construct
GGGGSGGGGSGGGGSGGGGSGGGGS

SEQ ID NO: 32 - protein, 5 aa, Synthetic construct
GGGGS

SEQ ID NO: 33 - protein, 117 aa, synthetic construct [sequence omitted]

SEQ ID NO: 34 - protein, 107 aa, synthetic construct [sequence omitted]

SEQ ID NO: 35 - protein, 8 aa, synthetic construct
GFTFSNFG

SEQ ID NO: 36 - protein, 8 aa, synthetic construct
ISGGGRDT

SEQ ID NO: 37 - protein, 10 aa, synthetic construct
VKWGNIYFDY

SEQ ID NO: 38 - protein, 6 aa, synthetic construct
LSINTF

SEQ ID NO: 39 - skipped

SEQ ID NO: 40 - protein, 9 aa, synthetic construct
QQSSNTPFT

SEQ ID NO: 41 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 214 aa, synthetic construct; identical to SEQ ID NO: 19

(19) United States
(12) Patent Application Publication
(10) Pub. No.: US 2025/0114468 A1
(43) Pub. Date: Apr. 10, 2025

(54) METHODS OF TREATING CANCER WITH A COMBINATION OF A PD1 INHIBITOR AND A TARGETED IMMUNOCYTOKINE

(71) Applicant: Regeneron Pharmaceuticals, Inc., Tarrytown, NY (US)

(72) Inventors: Jiaxi Wu, Pleasantville, NY (US); Ramandeep Bhavsar, Westwood, NJ (US)

(21) Appl. No.: 18/909,076

(22) Filed: Oct. 8, 2024

Related U.S. Application Data
(60) Provisional application No. 63/674,079, filed on Jul. 22, 2024.
(60) Provisional application No. 63/594,130, filed on Oct. 30, 2023.
(60) Provisional application No. 63/588,884, filed on Oct. 9, 2023.

(51) Int. Cl. A61K 47/68; A61P 35/00; C07K 16/28

(52) U.S. Cl. CPC A61K 47/6813; A61K 47/6849; A61P 35/00; C07K 16/2818; C07K 16/2827

(57) ABSTRACT

The present disclosure relates to methods of treating or inhibiting the growth of a tumor, wherein the methods include selecting a subject with cancer and administering to the subject in need thereof a therapeutically effective amount of a targeted immunocytokine (e.g., a fusion protein comprising an IL2 moiety and an immunoglobulin antigen-binding domain that binds to PD1) in combination with a PD1 inhibitor (e.g., an anti-PD1 antibody). The combination therapy demonstrates increased anti-tumor efficacy, increased duration of tumor control and/or increased overall survival of the subject, as compared to a subject administered the targeted immunocytokine as monotherapy.

### CROSS-REFERENCE TO RELATED APPLICATIONS

This application claims the benefit of priority to U.S. Provisional Patent Application No. 63/674,079 filed Jul. 22, 2024, U.S. Provisional Patent Application No. 63/594,130 filed Oct. 30, 2023, and U.S. Provisional Patent Application No. 63/588,884 filed Oct. 9, 2023, the disclosures of which are hereby incorporated by reference in their entireties.

### SEQUENCE LISTING

The sequence listing of the present application is submitted electronically as an ST.26 formatted xml file with a file name “SeqList-11608.xml,” creation date of Oct. 2, 2023, and a size of 40,070 bytes. This sequence listing submitted is part of the specification and is hereby incorporated by reference in its entirety. Sequences that are below a minimum length are AAS (SEQ ID NO: 7) and AAS (SEQ ID NO: 39).

### FIELD

The present disclosure relates to methods of treating or inhibiting the growth of a tumor by administering a combination of a targeted immunocytokine and a PD1 inhibitor.

### BACKGROUND

Cytokines are secreted proteins that can act in an autocrine and paracrine manner to activate and tune immune responses. Their immunomodulatory activities have provided a strong rationale for the exploration of cytokine-based therapeutics for cancer treatment (Waldmann, 2018, Cold Spring Harbor Perspectives in Biol., 10). The cytokine interleukin 2 (IL-2 or IL2) is a pluripotent cytokine produced primarily by activated T cells. It stimulates the proliferation and differentiation of T cells; induces the generation of cytotoxic T lymphocytes (CTLs) and the differentiation of peripheral blood lymphocytes to cytotoxic cells and lymphokine-activated killer (LAK) cells; promotes cytokine and cytolytic molecule expression by T cells; facilitates the proliferation and differentiation of B-cells and the synthesis of immunoglobulin by B-cells; and stimulates the generation, proliferation, and activation of natural killer (NK) cells.

IL-2 can signal through two types of receptor complexes formed from three subunits, IL-2Rα (IL2Ra, CD25), IL-2Rβ (IL2Rb, CD122), and IL-2Rγ (IL2Rg, CD132). The heterodimeric IL2Rb/g receptor binds to IL-2 with intermediate affinity and is mostly expressed on resting CD8+ T cells and NK cells; the heterotrimeric IL2Ra/b/g receptor binds to IL-2 with high affinity and is primarily expressed on Tregs and activated T cells (Spolski and Leonard, 2018, Nat. Rev. Immunol, 18:648-659). Recombinant IL-2 has been approved for the treatment of advanced melanoma and renal cell carcinoma and induced complete, durable tumor regression in some patients (Rosenberg, 2014, J. Immunol., 192:5451-5458). However, severe systemic toxicities associated with high-dose IL-2 therapy have limited its practical use (Dutcher et al., 2014, J. Immunotherapy of Cancer, 2:26; Vial and Descotes, 1992, Drug Safety, 7:417-433). To reduce toxicities and limit Treg expansion associated with high-dose IL-2 treatment, a new generation of IL-2 therapies with decreased binding to IL2Ra, and therefore biased activity towards IL2Rb/g dimeric receptor on CD8+ T and NK cells, have been developed (Charych et al., 2016, Clin. Cancer Res., 22:680-690; Klein et al., 2017, Oncoimmunology, 6: e1277306; Lopes et al., 2020, J. Immunother. Cancer, 8: e000673; Ptacin et al., 2021, Nat. Commun., 12 (1): 4785). While some of these molecules seem to deliver an improved safety profile in clinical trials, limited single agent efficacy has been reported from these studies (Bentebibel et al., 2019, Cancer Discovery, 9:711-721, Janku et al., 2021, Cancer Res., 81: LB041-LB041).

An alternative approach to improve efficacy and circumvent systemic toxicity of IL-2 is to convert it to an immunocytokine—an antibody-cytokine conjugate with the ability to target delivery of the cytokine to specific cell types. This could deliver IL-2 signaling specifically to T cells that are tumor-reactive. One cell-surface marker for such tumor-reactive T cells is the coinhibitory receptor programmed cell death protein 1 (PD-1 or PD1) (Gros et al., 2014, J. Clin. Investigation, 124:2246-2259; Gros et al., 2016, Nature Med., 22, 433-438). PD1 is expressed on the surface of antigen-activated T cells and antibodies that bind to PD1 may enable targeted delivery of IL-2. In this regard, PD1-targeted, IL2Ra-attenuated IL-2 mutein molecules have been recently developed to provide IL2R agonism preferentially to PD1⁺ tumor-reactive T cells (Codarri Deak et al., 2022, Nature, 610:161-172, Ren et al., 2022, J. Clin. Investigation, 132 (3): e153604).

A PD1 targeted, receptor masked IL-2 immunocytokine, termed anti-PD1-IL2Ra-IL2 (or “PD1-IL2Ra-IL2,” REGN10597) has been generated. Receptor masking should result in attenuated IL-2 activity for both IL2Ra/b/g trimeric and IL-2Rb/g dimeric receptors, while PD1-targeting should selectively reconstitute IL2Ra/b/g agonism on PD1⁺ tumor-reactive T cells. Monoclonal antibodies against PD1 are known in the art and have been described, for example, in U.S. Pat. Nos. 9,987,500, 8,008,449, 8,168,757, US20110008369, US20130017199, US 20130022595, WO 2006121168, WO 20091154335, WO 2012145493, WO 2013014668, WO 2009101611, EP 2262837, and EP 2504028. Cemiplimab (also known as REGN2810; LIBTAYO®), for example, is a high-affinity, fully human, hinge-stabilized IgG4^{P} antibody directed to the PD1 receptor that potently blocks the interaction of PD1 with its ligands, PD-L1 and PD-L2.

Despite the tremendous progress with PD1 checkpoint inhibitors in many cancer types, only a small subset of patients respond to single agent anti-PD1 therapy (Bagchi et al, 2021, Ann. Rev. Pathol.: Mechanisms of Disease, 16:223-249). Considerable efforts are being made to develop combination strategies with PD1 blockade to improve response rate and durability. One potential mechanism underlying the unresponsiveness to PD1 blockade is the paucity of tumor-infiltrating T cells (Teng et al., 2015, Cancer Res., 75:2139-2145; Vilain et al., 2017, Clin. Cancer Res., 23:5024-5033). T cells are major drivers of anti-cancer activity primarily through antigen-directed cytotoxicity against tumor cells (Waldman, 2020, Nat. Rev. Immunol., 20:651-668). IL2 is a key cytokine for T cell proliferation, differentiation, and effector function, and it has been used for the treatment of metastatic melanoma and renal cell carcinoma and induced complete, durable tumor regression in some patients. However, its broader use in cancer immunotherapy has been limited by severe toxicity.

There remains a need for more effective therapies in treating cancer that achieve substantially improved response without significant side effects.

### SUMMARY

In one aspect, the disclosed technology relates to a method for treating or inhibiting the growth of a tumor, including: (a) selecting a subject with cancer; and (b) administering to the subject a first therapy comprising a therapeutically effective amount of a targeted immunocytokine in combination with a second therapy comprising a therapeutically effective amount of a programmed death 1 (PD1) inhibitor, wherein administration of the combination leads to increased anti-tumor efficacy, as compared to a subject administered the targeted immunocytokine as monotherapy. In some embodiments, the targeted immunocytokine comprises a fusion protein comprising (i) an immunoglobulin (Ig) antigen-binding domain of a checkpoint inhibitor and (ii) an IL2 moiety. In some embodiments, the checkpoint inhibitor is an inhibitor of PD1, PD-L1, PD-L2, LAG-3, CTLA-4, TIM3, A2aR, B7H1, BTLA, CD160, LAIR1, TIGHT, VISTA, or VTCN1. In some embodiments, the checkpoint inhibitor is an inhibitor of PD1. In some embodiments, the IL2 moiety comprises: IL2 receptor alpha (IL2Ra) or a fragment thereof; and IL2 or a fragment thereof.

In some embodiments, the Ig antigen-binding domain of the checkpoint inhibitor comprises three heavy chain complementarity determining regions (CDRs) (HCDR1, HCDR2 and HCDR3) contained within a heavy chain variable region (HCVR) selected from the group consisting of SEQ ID NOs: 1, 11 and 20 and three light chain CDRs (LCDR1, LCDR2 and LCDR3) contained within a light chain variable region (LCVR) selected from the group consisting of SEQ ID NOs: 5 and 15. In some embodiments, the Ig antigen-binding domain of the checkpoint inhibitor comprises three heavy chain complementarity determining regions (CDRs) (HCDR1, HCDR2, and HCDR3) and three light chain CDRs (LCDR1, LCDR2, and LCDR3), wherein: (i) HCDR1, HCDR2, HCDR3, LCDR1, LCDR2, and LCDR3 comprise the amino acid sequences of SEQ ID NOs: 2, 3, 4, 6, 7, and 8, respectively; (ii) HCDR1, HCDR2, HCDR3, LCDR1, LCDR2, and LCDR3 comprise the amino acid sequences of SEQ ID NOs: 12, 13, 14, 16, 7, and 17, respectively; or (iii) HCDR1, HCDR2, HCDR3, LCDR1, LCDR2, and LCDR3 comprise the amino acid sequences of SEQ ID NOs: 21, 22, 23, 6, 7, and 8, respectively.

In some embodiments, the Ig antigen-binding domain of the checkpoint inhibitor comprises a HCVR/LCVR amino acid sequence pair selected from SEQ ID NOs: 1/5, 11/15, and 20/5. In some embodiments, the Ig antigen-binding domain of the checkpoint inhibitor comprises a HCVR/LCVR amino acid sequence pair of SEQ ID NOs: 20/5. In some embodiments, the fusion protein comprises a heavy chain comprising a HCVR and a heavy chain constant region of IgG1 or IgG4 isotype. In some embodiments, the fusion protein comprises a heavy chain and a light chain, wherein: (i) the heavy chain comprises the amino acid sequence of SEQ ID NO: 24, and the light chain comprises the amino acid sequence of SEQ ID NO: 25; (ii) the heavy chain comprises the amino acid sequence of SEQ ID NO: 9, and the light chain comprises the amino acid sequence of SEQ ID NO: 10; or (iii) the heavy chain comprises the amino acid sequence of SEQ ID NO: 18, and the light chain comprises the amino acid sequence of SEQ ID NO: 19.

In some embodiments, the IL2 moiety is attached to the C-terminus of the heavy chain via a linker comprising the amino acid sequence of SEQ ID NO: 30 or 31. In some embodiments, the IL2 moiety comprises IL2 having the amino acid sequence of SEQ ID NO: 29. In some embodiments, the IL2 moiety comprises the IL2 or fragment thereof connected via a linker to the C-terminus of the IL2Ra or fragment thereof. In some embodiments, the IL2Ra or fragment thereof comprises the amino acid sequence of SEQ ID NO: 28. In some embodiments, the IL2 moiety comprises the amino acid sequence of SEQ ID NO: 27. In some embodiments, the PD1 inhibitor of the second therapy comprises an antibody or antigen-binding fragment thereof that binds specifically to PD1, PD-L1 or PD-L2, or a bioequivalent thereof. In some embodiments, the PD1 inhibitor of the second therapy is an anti-PD1 antibody selected from cemiplimab, nivolumab, pembrolizumab, pidilizumab, MEDI0608, BI 754048, PF-06371548, spartalizumab, camrelizumab, JNJ-63313240, and MCLA-134. In some embodiments, the PD1 inhibitor of the second therapy is an anti-PD-L1 antibody selected from REGN3504, avelumab, atezolizumab, durvalumab, MDX-1105, LY3300054, FAZ053, STI-1014, CX-031, KN035, and CK-301.

In some embodiments, the PD1 inhibitor of the second therapy is an antibody or antigen-binding fragment thereof that binds specifically to PD1 and comprises three heavy chain complementarity determining regions (CDRs) (HCDR1, HCDR2 and HCDR3) contained within a heavy chain variable region (HCVR) of SEQ ID NO: 33 and three light chain CDRs (LCDR1, LCDR2 and LCDR3) contained within a light chain variable region (LCVR) of SEQ ID NO: 34. In some embodiments, the HCDR1, HCDR2, HCDR3, LCDR1, LCDR2, and LCDR3 of the anti-PD1 antibody or antigen-binding fragment thereof of the second therapy comprise the amino acid sequences of SEQ ID NOs: 35, 36, 37, 38, 39, and 40, respectively. In some embodiments, the antibody or antigen-binding fragment thereof of the second therapy that binds specifically to PD1 comprises a HCVR comprising the amino acid sequence of SEQ ID NO: 33 and/or a LCVR comprising the amino acid sequence of SEQ ID NO: 34. In some embodiments, the antibody of the second therapy that binds specifically to PD1 comprises a heavy chain and a light chain, wherein the heavy chain has an amino acid sequence of SEQ ID NO: 41 and/or the light chain has an amino acid sequence of SEQ ID NO: 42. In some embodiments, the PD1 inhibitor of the second therapy comprises is cemiplimab or a bioequivalent thereof.

In some embodiments, the cancer is selected from anal cancer, angiosarcoma, basal cell carcinoma, bladder cancer, bone cancer, brain cancer, breast cancer, cervical cancer, cholangiocarcinoma, chondrosarcoma, colon cancer, colorectal cancer, cutaneous squamous cell carcinoma, endometrial cancer, esophageal cancer, glioblastoma multiforme, head and neck squamous cell cancer, hepatocellular carcinoma, kidney cancer, liver cancer, lung cancer, Merkel cell carcinoma, melanoma, myeloma, non-small cell lung cancer, ovarian cancer, pancreatic cancer, prostate cancer, salivary gland cancer, skin cancer, soft tissue sarcoma, stomach cancer, testicular cancer, and uterine cancer.

In some embodiments, the targeted immunocytokine is administered to the subject before or concurrently with the administration of the PD1 inhibitor. In some embodiments, the targeted immunocytokine is administered to the subject after the administration of the PD1 inhibitor.

In another aspect, the disclosed technology relates to a method for increasing the efficacy of targeted immunocytokine therapy, comprising: (a) selecting a subject with cancer; and (b) administering to the subject a first therapy comprising a therapeutically effective amount of a targeted immunocytokine in combination with a second therapy comprising a therapeutically effective amount of a programmed death 1 (PD1) inhibitor, wherein administration of the combination leads to increased anti-tumor efficacy, as compared to a subject administered the targeted immunocytokine as monotherapy. In some embodiments, the targeted immunocytokine comprises a fusion protein comprising (a) an immunoglobulin antigen-binding domain comprising a heavy chain variable region having an amino acid sequence of SEQ ID NO: 20 and a light chain variable region having an amino acid sequence of SEQ ID NO: 5; and (b) an IL2 moiety comprising IL2 or a fragment thereof connected via a linker to the C-terminus of IL2Ra or fragment thereof. In some embodiments, the PD1 inhibitor of the second therapy is an antibody or antigen-binding fragment thereof that binds specifically to PD1 and comprises three heavy chain complementarity determining regions (CDRs) (HCDR1, HCDR2 and HCDR3) contained within a heavy chain variable region (HCVR) of SEQ ID NO: 33 and three light chain CDRs (LCDR1, LCDR2 and LCDR3) contained within a light chain variable region (LCVR) of SEQ ID NO: 34.

Other embodiments of the present disclosure will become apparent from the detailed description below.

### BRIEF DESCRIPTION OF THE DRAWINGS

FIGS. 1A-1D relate to Example 2 and show the requirement of engagement of IL-2Ra for anti-tumor activity of IL-2. FIG. 1A is a graph showing levels of STAT5 phosphorylation on gated Tregs as measured by flow cytometry. FIG. 1B is a graph showing levels of STAT5 phosphorylation on CD8+ T cells as measured by flow cytometry. FIG. 1C is a graph showing levels of STAT5 phosphorylation on NK cells as measured by flow cytometry.

FIG. 1D is a graph showing levels of IL-2Ra expression on each gated population as measured by flow cytometry.

FIGS. 2A-2C relate to Example 2. FIG. 2A is a diagram showing the design of the in vivo experiment. FIG. 2B is a graph showing average tumor growth. FIG. 3C is a graph showing survival in the experiment.

FIGS. 3A-3B relate to Example 2 and are graphs showing SPR sensorgrams for binding of PD1-IL2Ra-IL2 or control molecules to the indicated IL-2 receptor subunit proteins. FIG. 3A is a graph showing binding to IL-2 receptor subunit protein IL-2Ra. FIG. 3B is a graph showing binding to IL-2 receptor subunit proteins IL-2Rb and IL-2Rg.

FIGS. 4A-4B relate to Example 2 and are graphs showing binding to cells analyzed by flow cytometry. Insets are magnifications of the designated portion of the larger graph. FIG. 4A is a graph showing a titration of each indicated molecule binding to YT/STAT5-Luc/IL2Ra^{KO}/PD1^{KO} cells. FIG. 4B is a graph showing a titration of each indicated molecule binding to YT/STAT5-Luc/IL2Ra^{OE}/PD1^{KO} cells.

FIGS. 5A-5C relate to Example 2 and are graphs showing activation of a STAT5 luciferase reporter on three YT/STAT5-Luc/IL2Ra^{KO} cell lines expressing specific levels of PD-1 by a titration of each indicated molecule. FIG. 5A is a graph showing activation on a YT/STAT5-Luc/IL2Ra^{KO} cell line expressing zero PD-1. FIG. 5B is a graph showing activation on a YT/STAT5-Luc/IL2Ra^{KO} cell line expressing an intermediate level of PD-1. FIG. 5C is a graph showing activation on a YT/STAT5-Luc/IL2Ra^{KO} cell line expressing a high level of PD-1.

FIGS. 6A-6B relate to Example 2 and are graphs showing levels of STAT5 phosphorylation measured by flow cytometry. FIG. 6A is a graph showing levels of STAT5 phosphorylation on gated PD1⁻ CD8⁺ T cells. FIG. 6B is another graph showing levels of STAT5 phosphorylation on gated PD1⁺ CD8⁺ T cells.

FIGS. 7A-7B relate to Example 2 and are graphs showing T cell counts among peripheral blood leukocytes per ml of blood analyzed by flow cytometry. FIG. 7A is a graph showing counts of PD1⁻ CD8⁺ T cells per ml of blood. FIG. 7B is a graph showing counts of PD1⁺ CD8⁺ T cells per ml of blood.

FIGS. 8A-8B relate to Example 2. FIG. 8A is a graph showing change in body weight. FIG. 8B is a graph showing change in pulmonary wet weight.

FIGS. 9A-9D relate to Example 2 and are graphs showing the number of tumor free (TF) mice at end of study and average tumor volume in mice administered PD1-IL2Ra-IL2 in different syngeneic tumor models. FIG. 9A shows the number of TF mice and average tumor volume of mice implanted with B16F10 tumor cells. FIG. 9B shows the number of TF mice and average tumor volume of mice implanted with MCA205 tumor cells. FIG. 9C shows the number of TF mice and average tumor volume of mice implanted with TRAMP-C2 tumor cells. FIG. 9D shows the number of TF mice and the average tumor volume of mice implanted with Colon26 tumor cells.

FIGS. 10A-10B relate to Example 2. FIG. 10A is a graph showing average tumor size. FIG. 10B is a graph showing survival.

FIGS. 11A-11C relate to Example 2. FIG. 11A is a schematic of the in vivo experiment. FIG. 11B is a graph showing average tumor size for the experiment. FIG. 11C is a graph showing survival for the experiment.

FIG. 12 relates to Example 2 and is a graph showing average tumor volume.

FIGS. 13A-13E relate to Example 2 and are graphs showing T cell densities in excised tumors of mice administered the indicated molecules. FIG. 13A is a graph showing tumor CD8⁺ T cell density. FIG. 13B is a graph showing tumor SPAS-1-Dextramer⁺ CD8⁺ T cells as a percentage of total CD8⁺ T cells. FIG. 13C is a graph showing tumor SPAS-1-Dextramer⁺ CD8⁺ T cell density. FIG. 13D is a graph showing tumor PD1⁺ CD8⁺ T cell density. FIG. 13E is a graph showing tumor TCF1⁺PD1⁺ CD8⁺ T cell density.

FIG. 14 relates to Example 3 and is a graph showing average tumor volume of mice administered the indicated molecules.

FIGS. 15A-15D relate to Example 3 and are graphs showing individual tumor growth curves and the number of tumor free mice at the end of the same study as in FIG. 14. Arrow indicates mice that initially underwent tumor regression. FIG. 15A shows individual tumor growth curves and the number of tumor free mice of mice administered Isotype A and Isotype B. FIG. 15B shows individual tumor growth curves and the number of tumor free mice of mice administered Isotype A and anti-PD1. FIG. 15C shows individual tumor growth curves and the number of tumor free mice of mice administered anti-PD1-IL2Ra-IL2 and Isotype B. FIG. 15D shows individual tumor growth curves and the number of tumor free mice of mice administered anti-PD1-IL2Ra-IL2 and anti-PD1.

FIG. 16 relates to Example 3 and is a graph showing survival of mice in the same study as in FIG. 14.

FIG. 17 relates to Example 3 and is a graph showing body weight change over time of mice administered the indicated antibodies.

FIG. 18 relates to Example 5 and is a set of graphs showing STAT3 activity of the indicated cell lines treated with the indicated molecules.

FIG. 19 relates to Example 5 and is a schematic of the in vivo experiment.

FIGS. 20A-20H relate to Example 5 and are graphs showing the concentration of the indicated cell types in tumors harvested from mice administered PBS or mPD1-IL2Ra-IL2. FIG. 20A is a graph showing the concentration of Total T cells. FIG. 20B is a graph showing the percent of Total T cells that are CD8 or CD4 T cells. FIG. 20C is a graph showing the concentration of CD8 T cells. FIG. 20D is a graph showing the concentration of PD-1+ CD8 T cells. FIG. 20E is a graph showing the concentration of CD4 T cells. FIG. 20F is a graph showing the concentration of PD-1+ CD4 T cells. FIG. 20G is a graph showing the concentration of Tregs. FIG. 20H is a graph showing the concentration of PD-1+ Tregs.

FIGS. 21A-21H relate to Example 5 and are graphs showing the concentration of the indicated cell types in spleens harvested from mice administered PBS or mPD1-IL2Ra-IL2. FIG. 21A is a graph showing the number of Total T cells per spleen. FIG. 21B is a graph showing the percent of Total T cells that are CD8 or CD4 T cells. FIG. 21C is a graph showing the number of CD8 T cells per spleen. FIG. 21D is a graph showing the number of PD-1+ CD8 T cells per spleen. FIG. 21E is a graph showing the number of CD4 T cells per spleen. FIG. 20F is a graph showing the number of PD-1+ CD4 T cells per spleen. FIG. 21G is a graph showing the number of Tregs per spleen. FIG. 21H is a graph showing the number of PD-1+ Tregs per spleen.

FIG. 22 relates to Example 5 and is a set of graphs showing the ratio of CD8 T cells to Treg cells from tumors and spleens of mice administered PBS or mPD1-IL2Ra-IL2.

### DETAILED DESCRIPTION

It is to be understood that the present disclosure is not limited to the particular methods and experimental conditions described, as such methods and conditions may vary. It is also to be understood that the terminology used herein is for the purpose of describing particular embodiments only, and is not intended to be limiting, and that the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are hereby incorporated by reference in their entirety unless otherwise stated.

As used herein, the singular forms “a,” “an,” and “the” include plural reference unless the context clearly dictates otherwise; and the terms “and/or” or “/” means any one of the items, any combination of the items, or all of the items with which this term is associated.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

### Methods of Treating or Inhibiting the Growth of a Tumor

The present disclosure includes methods of treating or inhibiting the growth of a tumor by administering to a subject in need thereof a combination therapy comprising a therapeutically effective amount of a targeted immunocytokine and a therapeutically effective amount of a PD1 inhibitor. The present disclosure also includes methods of increasing the efficacy of a targeted immunocytokine by administering to a subject with cancer a combination therapy comprising a therapeutically effective amount of a targeted immunocytokine and a therapeutically effective amount of a PD1 inhibitor.

Targeted immunocytokines of the present disclosure include fusion proteins comprising an antigen-binding moiety that specifically binds to a checkpoint inhibitor (e.g., human PD1) that targets tumor-reactive T cells. The antigen-binding moiety is helpful in selectively reconstituting activity on the tumor-reactive T cells. Without this targeting moiety, a non-targeted (NT) immunocytokine would be less effective at specifically activating tumor-reactive T cells and could induce undesirable on-target, off-tumor effects. In some embodiments, targeted immunocytokines of the present disclosure further comprise a cytokine moiety that includes a cytokine (e.g., IL2) bound to a subunit of a receptor of the cytokine (e.g., IL2Ra) or a fragment of the receptor subunit. The targeted immunocytokine, however, retains the ability to engage the endogenous receptor (e.g., the high-affinity heterotrimeric IL2Ra/b/g receptor) which is required for anti-tumor efficacy. In some embodiments, targeted immunocytokines of the present disclosure may form homodimers via their respective heavy chain constant regions, which allows the cytokine of one subunit to be bound by the receptor of the other subunit, known as a trans-sequestered conformation. This maintains engagement with activated CD8+ T cells; however, the bound configuration of the cytokine moiety helps in masking of the cytokine and attenuating its activity, thus leading to reduced systemic toxicity. By specifically activating T cells that are tumor-reactive, the targeted immunocytokines of the present disclosure promote effective immunological control of tumor growth.

As used herein, the terms “treating,” “treat,” or the like, mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for surgery, and/or to increase duration of survival of the subject. In some embodiments, the terms “tumor,” “lesion,” and “cancer” are used interchangeably and refer to one or more cancerous growths.

As used herein, the expression “a subject in need thereof” means a human or non-human mammal that exhibits one or more symptoms or indications of cancer and/or who has been diagnosed with cancer, and who needs treatment for the same. In some embodiments, the terms “subject” and “patient” are used interchangeably. The expression also includes patients with primary, established, metastatic, or recurrent tumors (advanced malignancies). In certain embodiments, the expression “a subject in need thereof” includes a subject with a tumor that is resistant to or refractory to or is inadequately controlled by prior therapy (e.g., treatment with an anti-cancer agent).

In certain embodiments, the methods of the present disclosure are used for treating a subject with a solid tumor. As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumors. The term includes different types of solid tumors named for the cell types that form them, viz. sarcomas, carcinomas and blastomas. In certain embodiments, the term “solid tumor” refers to cancers including, but not limited to, anal cancer, angiosarcoma, basal cell carcinoma, bladder cancer, bone cancer, brain cancer, breast cancer, cervical cancer, cholangiocarcinoma, chondrosarcoma, colon cancer, colorectal cancer, cutaneous squamous cell carcinoma, endometrial cancer, esophageal cancer, glioblastoma multiforme, head and neck squamous cell cancer, hepatocellular carcinoma, kidney cancer, liver cancer, lung cancer, Merkel cell carcinoma, melanoma, myeloma, non-small cell lung cancer, ovarian cancer, pancreatic cancer, prostate cancer, salivary gland cancer, skin cancer, soft tissue sarcoma, stomach cancer, testicular cancer, and uterine cancer.

In certain embodiments, the disclosed methods include administering therapeutically effective amounts of each of a targeted immunocytokine and a PD1 inhibitor (e.g., cemiplimab or a bioequivalent thereof) in combination with an additional therapeutic agent or therapy. The additional therapeutic agent or therapy may include one or more of a NSAID, a corticosteroid, an antibody to a tumor specific antigen, an IDO inhibitor, an Ang2 inhibitor, a cancer vaccine, an oncolytic virus, an EGFR inhibitor, a TGF-beta inhibitor, an antibody to PD-L1, a CTLA-4 inhibitor, a LAG3 inhibitor, a TIM3 inhibitor, a dietary supplement, a VEGF antagonist, surgery, radiation, a chemotherapeutic agent, and a cytotoxic agent.

In some embodiments, the disclosed methods lead to increased efficacy and duration of anti-tumor response. Methods according to this aspect of the disclosure comprise selecting a subject with cancer and administering to the subject a therapeutically effective amount of a targeted immunocytokine in combination with a therapeutically effective amount of a PD1 inhibitor. In some embodiments, the disclosed methods provide for increased tumor inhibition and/or increased duration of the anti-tumor response, e.g., by about 20%, more than 20%, more than 30%, more than 40%, more than 50%, more than 60%, more than 70%, or more than 80% as compared to an untreated subject or a subject treated with either the targeted immunocytokine or PD1 inhibitor as monotherapy. In some embodiments, the disclosed methods increase duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% more than an untreated subject or a subject treated with either the targeted immunocytokine or PD1 inhibitor as monotherapy.

In some embodiments, administration of the disclosed combination therapy leads to one or more of: (i) delay in tumor growth and development; (ii) increased disease-free survival (DFS) from date of treatment until recurrence of tumor or death; and (iii) improved overall response rate (ORR), complete response (CR; complete disappearance of all evidence of tumor cells), partial response (PR; at least 30% or more decrease in tumor cells or tumor size), increased overall survival (OS), or increased progression-free survival (PFS), each as compared to an untreated subject or a subject treated with either the targeted immunocytokine or PD1 inhibitor as monotherapy. Tumor reduction can be measured by known methods, e.g., X-rays, positron emission tomography (PET), computed tomography (CT), magnetic resonance imaging (MRI), cytology, histology, or molecular genetic analyses.

### Targeted Immunocytokines

As used herein, a “targeted immunocytokine” refers to a cytokine such as interleukin 2 (IL2) that is linked to a moiety that binds to an immune checkpoint (i.e., “targets” an immune checkpoint). Non-limiting examples of the immune checkpoint inhibitor include inhibitors of PD1, PD-L1, PD-L2, LAG-3, CTLA-4, TIM3, A2aR, B7H1, BTLA, CD160, LAIR1, TIGHT, VISTA, or VTCN1. In some embodiments, the targeted immunocytokine includes an immunoglobulin antigen-binding domain that binds specifically to an immune checkpoint. In one preferred embodiment, the checkpoint inhibitor is an inhibitor of PD1 (e.g., an anti-PD1 antibody or antigen-binding fragment thereof).

In certain embodiments, the targeted immunocytokine is a fusion protein that includes (i) an antigen-binding domain of a checkpoint inhibitor and (ii) an IL2 moiety. In some embodiments, the antigen-binding domain binds specifically to human PD1. In some embodiments, the antigen-binding domain is an antibody or antigen-binding fragment thereof. In some embodiments, the targeted immunocytokine is an anti-PD1-IL2Ra-IL2 fusion protein.

As used herein, the term “fusion protein” means a protein comprising two or more polypeptide sequences that are joined together covalently or non-covalently. Fusion proteins encompassed by the present disclosure may include translation products of a chimeric gene construct that joins the nucleic acid sequences encoding a first polypeptide with the nucleic acid sequence encoding a second polypeptide to form a single open reading frame. Alternatively, the fusion protein may be encoded by two or more gene constructs on separate vectors that may be co-expressed in a host cell. In general, a “fusion protein” is a recombinant protein of two or more proteins joined by a peptide bond or by several peptides. In some embodiments, the fusion protein may also comprise a peptide linker between the two domains.

Fusion proteins disclosed herein may include one or more conservative modifications. A fusion protein with one or more conservative modifications may retain the desired functional properties, which can be tested using the functional assays known in the art. The term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine). includes one or more conservative modifications. The Cas protein with one or more conservative modifications may retain the desired functional properties, which can be tested using the functional assays known in the art. As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine).

As used herein, an “antibody” refers to an immunoglobulin molecule comprised of four polypeptide chains, two heavy (H) chains and two light (L) chains inter-connected by disulfide bonds (i.e., “full antibody molecules”), as well as multimers thereof (e.g., IgM) or antigen-binding fragments thereof. Each heavy chain is comprised of a heavy chain variable region (“HCVR” or “VH”) and a heavy chain constant region (comprised of domains CH1, CH2, and CH3). Each light chain is comprised of a light chain variable region (“LCVR or “VL”) and a light chain constant region (CL). The VH and VL regions can be further subdivided into regions of hypervariability, termed CDRs, interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding fragment thereof) may be identical to the human germline sequences or may be naturally or artificially modified. An amino acid consensus sequence may be defined based on a side-by-side analysis of two or more CDRs. The term “antibody” also includes antigen-binding fragments of full antibody molecules.

As used herein, an “antigen” refers to any substance that causes the immune system to produce antibodies or specific cell-mediated immune responses against it. A disease-associated antigen is any substance that is associated with any disease that causes the immune system to produce antibodies or a specific cell-mediated response against it.

As used herein, the “antigen-binding fragment” of an antibody, “antigen-binding portion” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated CDR, such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

In some embodiments, the antigen-binding domain of the fusion protein comprises three heavy chain CDRs (HCDR1, HCDR2, and HCDR3) and three light chain CDRs (LCDR1, LCDR2, and LCDR3), wherein: HCDR1 comprises an amino acid sequence of SEQ ID NO: 2, 12, or 21; HCDR2 comprises an amino acid sequence of SEQ ID NO: 3, 13, or 22; HCDR3 comprises an amino acid sequence of SEQ ID NO: 4, 14, or 23; LCDR1 comprises an amino acid sequence of SEQ ID NO: 6 or 16; LCDR2 comprises an amino acid sequence of SEQ ID NO: 7; and LCDR3 comprises an amino acid sequence of SEQ ID NO: 8 or 17. In some embodiments, the antigen-binding domain comprises HCDR1, HCDR2, HCDR3, LCDR1, LCDR2, and LCDR3 comprising respective amino acid sequences of (i) SEQ ID NOs: 2, 3, 4, 6, 7, and 8; (ii) SEQ ID NOs: 12, 13, 14, 16, 7, and 17; or (iii) SEQ ID NOs: 21, 22, 23, 6, 7, and 8. In some embodiments, the antigen-binding domain comprises HCDR1, HCDR2, HCDR3, LCDR1, LCDR2, and LCDR3 comprising the amino acid sequences of SEQ ID NOs: 21, 22, 23, 6, 7, and 8, respectively. In some embodiments, the antigen-binding domain comprises a HCVR/LCVR amino acid sequence pair selected from SEQ ID NOs: 1/5, 11/15, and 20/5. In one embodiment, the antigen-binding domain comprises a HCVR/LCVR amino acid sequence pair of SEQ ID NOs: 20/5.

In some embodiments, the antigen-binding domain of the fusion protein comprises a heavy chain/light chain sequence pair comprising the amino acid sequences of SEQ ID NOs: 9/10, 18/19, or 24/25. In some embodiments, the fusion protein comprises a heavy chain/light chain sequence pair comprising the amino acid sequences of SEQ ID NOs: 24 and 25.

In some embodiments, the IL2 moiety comprises (i) IL2 or a fragment thereof; and (ii) IL2 receptor alpha (“IL2Ra” or “IL2Ra”) or a fragment thereof. In some embodiments, the IL2 moiety may include a wild type (e.g., human wild type) or variant IL2 domain that is fused to an IL2 binding domain of IL2Ra, optionally via a linker. In some embodiments, the IL2 binding domain of IL2Ra of a fragment thereof is bound at its C-terminus via a linker to the IL2 (wild type or variant) domain or fragment thereof. As used herein, a “wild-type” form of IL2 is a form of IL2 that is otherwise the same as a mutant IL2 polypeptide except that the wild-type form has a wild-type amino acid at each amino acid position of the mutant IL2 polypeptide. For example, if the IL2 mutant is the full-length IL2 (i.e., IL2 not fused or conjugated to any other molecule), the wild-type form of this mutant is full-length native IL2.

In some embodiments, the IL2 or fragment thereof comprises the amino acid sequence of SEQ ID NO: 29. In some embodiments, the IL2 moiety comprises the amino acid sequence of SEQ ID NO: 27.

The targeted immunocytokine may include one or more linkers (e.g., peptide linker or non-peptide linker) connecting the various components of the molecule. In some embodiments, two or more components of the targeted immunocytokine are connected to one another by a peptide linker. By way of a non-limiting example, linkers can be used to connect (a) an IL2 moiety and an antigen-binding domain of a checkpoint inhibitor; (b) different domains within an IL2 moiety (e.g., an IL2 domain and an IL2Ra domain); or (c) different domains within an antigen-binding moiety (e.g., different components of anti-PD1 antigen-binding domain).

Examples of flexible linkers that may be used in the disclosed targeted immunocytokine include those disclosed in Chen et al., Adv Drug Deliv Rev., 65 (10): 1357-69 (2013) and Klein et al., Protein Engineering, Design & Selection, 27 (10): 325-30 (2014). Particularly useful flexible linkers are or comprise repeats of glycines and serines, e.g., a monomer or multimer of GnS or SGn, where n is an integer from 1 to 10, e.g., 1, 2, 3, 4, 5, 6, 7, 8, 9, or 10. In some embodiments, the linker is or comprises a monomer or multimer of repeating G4S (GGGGS; SEQ ID NO: 32), e.g., (GGGGS)ₙ.

In some embodiments, the IL2 moiety and the antigen-binding moiety are connected via a linker that comprises an amino acid sequence of one or more repeats of GGGGS (SEQ ID NO: 32). In some embodiments, the linker comprises an amino acid sequence of SEQ ID NO: 30 or 31. In some embodiments, the IL2 moiety is linked to the C-terminus of the antigen-binding moiety via a peptide linker. In some embodiments, the linker comprises an amino acid sequence of SEQ ID NO: 30.

The targeted immunocytokine of the present disclosure exhibits attenuated binding to IL2Rα, IL2Rβ and IL2Rγ. In some embodiments, the targeted immunocytokine does not compete with cemiplimab, pembrolizumab or nivolumab. In some embodiments, the targeted immunocytokine exhibits reduced activity in activating human IL2Rα/β/γ trimeric and IL2Rβ/γ dimeric receptor complexes as compared to IL2 and increased activity in activating human IL2Rα/β/γ trimeric and IL2Rβ/γ dimeric receptor complexes as compared to a non-targeted IL2Rα-IL2 construct. In some embodiments, the targeted immunocytokine exhibits increased activity in stimulating antigen-activated T cells as measured by a level of IFN-γ release as compared to a wild type human IL2.

### PD1 Inhibitors

As used herein, a “PD1 inhibitor” refers to any molecule capable of inhibiting, blocking, abrogating or interfering with the activity or expression of PD1. In some embodiments, the PD1 inhibitor can be an antibody, a small molecule compound, a nucleic acid, a polypeptide, or a functional fragment or variant thereof. Non-limiting examples of suitable PD1 inhibitor antibodies include anti-PD1 antibodies and antigen-binding fragments thereof, anti-PD-L1 antibodies and antigen-binding fragments thereof, and anti-PD-L2 antibodies and antigen-binding fragments thereof. Other non-limiting examples of suitable PD1 inhibitors include RNAi molecules such as anti-PD1 RNAi molecules, anti-PD-L1 RNAi, and an anti-PD-L2 RNAi, antisense molecules such as anti-PD1 antisense RNA, anti-PD-L1 antisense RNA, and anti-PD-L2 antisense RNA, and dominant negative proteins such as a dominant negative PD1 protein, a dominant negative PD-L1 protein, and a dominant negative PD-L2 protein. Some examples of the foregoing PD1 inhibitors are described in e.g., U.S. Pat. Nos. 9,308,236, 10,011,656, and US20170290808.

The antibodies used in the methods disclosed herein may be human antibodies. As used herein, the term “human antibody” refers to antibodies having variable and constant regions derived from human germline immunoglobulin sequences. The human antibodies of the present disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods disclosed herein may be recombinant human antibodies. As used herein, the term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In some embodiments, PD1 inhibitors used in the methods disclosed herein are antibodies or antigen-binding fragments thereof that specifically bind PD1. In some embodiments, the term “anti-PD1 antibody or antigen-binding fragment thereof” is used interchangeably with the term “antibody or antigen-binding fragment thereof that specifically binds PD1.” The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like.

In some embodiments, the PD1 inhibitor is a bioequivalent of an anti-PD1 antibody or antigen-binding fragment thereof. As used herein, the term “bioequivalent” refers to anti-PD1 antibodies or PD1-binding proteins or fragments thereof that are pharmaceutical equivalents or pharmaceutical alternatives whose rate and/or extent of absorption do not show a significant difference with that of a reference antibody (e.g., cemiplimab) when administered at the same molar dose under similar experimental conditions, either single dose or multiple dose. In the context of the present disclosure, the term “bioequivalent” includes antigen-binding proteins that bind to PD1 and do not have clinically meaningful differences with the reference antibody (e.g., cemiplimab) with respect to safety, purity and/or potency.

According to certain embodiments, the PD1 inhibitor is an anti-PD1 antibody including three heavy chain complementarity determining regions (HCDRs) of a heavy chain variable region (HCVR) including the amino acid sequence of SEQ ID NO: 33 and three light chain complementarity determining regions (LCDRs) of a light chain variable region (LCVR) including the amino acid sequence of SEQ ID NO: 34. According to certain embodiments, the anti-PD1 antibody includes three HCDRs (HCDR1, HCDR2 and HCDR3) and three LCDRs (LCDR1, LCDR2 and LCDR3), wherein the HCDR1 includes the amino acid sequence of SEQ ID NO: 35; the HCDR2 includes the amino acid sequence of SEQ ID NO: 36; the HCDR3 includes the amino acid sequence of SEQ ID NO: 37; the LCDR1 includes the amino acid sequence of SEQ ID NO: 38; the LCDR2 includes the amino acid sequence of SEQ ID NO: 39; and the LCDR3 includes the amino acid sequence of SEQ ID NO: 40. In certain embodiments, the anti-PD1 antibody includes an HCVR including SEQ ID NO: 33 and an LCVR including SEQ ID NO: 34. In certain embodiments, the anti-PD1 antibody includes a heavy chain including the amino acid sequence of SEQ ID NO: 41 and a light chain including the amino acid sequence of SEQ ID NO: 42. An exemplary anti-PD1 antibody comprising an HCVR of SEQ ID NO: 33 and an LCVR of SEQ ID NO: 34 for use in the disclosed methods is cemiplimab.

Other anti-PD1 antibodies that can be used in the context of the methods of the present disclosure include, e.g., the antibodies referred to and known in the art as nivolumab, pembrolizumab, MEDI0608, pidilizumab, BI 754091, spartalizumab (also known as PDR001), camrelizumab (also known as SHR-1210), JNJ-63723283, MCLA-134, or any of the anti-PD1 antibodies set forth in U.S. Pat. Nos. 6,808,710, 7,488,802, 8,008,449, 8,168,757, 8,354,509, 8,609,089, 8,686,119, 8,779,105, 8,900,587, and 9,987,500, and in patent publications WO2006/121168, WO2009/114335. The portions of all of the aforementioned publications that identify anti-PD1 antibodies are hereby incorporated by reference.

According to certain embodiments, a bioequivalent of cemiplimab is an anti-PD1 antibody including a HCVR having 90%, 95%, 98% or 99% sequence identity to SEQ ID NO: 33. According to certain embodiments, a bioequivalent of cemiplimab is an anti-PD1 antibody including a LCVR having 90%, 95%, 98% or 99% sequence identity to SEQ ID NO: 34. According to certain embodiments, a bioequivalent of cemiplimab is an anti-PD1 antibody including a HCVR having 90%, 95%, 98% or 99% sequence identity to SEQ ID NO: 33, and a LCVR having 90%, 95%, 98% or 99% sequence identity to SEQ ID NO: 34. Sequence identity may be measured by methods known in the art (e.g., GAP, BESTFIT, and BLAST).

In some embodiments, PD1 inhibitors used in the methods disclosed herein are antibodies or antigen-binding fragments thereof that specifically bind PD-L1. Non-limiting examples of anti-PD-L1 antibodies for use in the disclosed methods include REGN3504, MDX-1105, atezolizumab (TECENTRIQ™), durvalumab (IMFINZI™), avelumab (BAVENCIO™), LY3300054, FAZ053, STI-1014, CX-072, KN035 (Zhang et al., Cell Discovery, 3, 170004 (March 2017)), CK-301 (Gorelik et al., American Association for Cancer Research Annual Meeting (AACR), 2016 Apr. 4 Abstract 4606), and other known anti-PD-L1 antibodies. See U.S. Pat. Nos. 7,943,743, 8,217,149, 9,402,899, 9,624,298, 9,938,345, WO 2007005874, WO 2010077634, WO 2013181452, WO 2013181634, WO 2016149201, WO 2017034916, or EP3177649.

### Combination Therapies

In general, the methods of the present disclosure include administering a therapeutically effective amount of a targeted immunocytokine in combination with a therapeutically effective amount of a PD1 inhibitor. As used herein, the expression “in combination with” means that the targeted immunocytokine is administered before, after, or concurrently with the PD1 inhibitor. This expression includes sequential or concurrent administration of the targeted immunocytokine and PD1 inhibitor.

In some embodiments, the disclosed combination therapy treats or inhibits the growth of a tumor in a subject. In some embodiments, administration of the disclosed PD1 inhibitor increases the efficacy of the targeted immunocytokine. In some embodiments, the combined administration of the targeted immunocytokine and PD1 inhibitor with an additional therapeutic agent or therapy leads to improved anti-tumor efficacy, reduced side effects of one or both of the primary therapies, and/or reduced dosage of one or both of the primary therapies.

In some embodiments, the disclosed methods further include administration of an additional therapeutic agent or therapy. Non-limiting examples of the additional therapeutic agent or therapy include one or more of a NSAID, a corticosteroid, an antibody to a tumor specific antigen, an IDO inhibitor, an Ang2 inhibitor, a cancer vaccine, an oncolytic virus, an EGFR inhibitor, a TGF-beta inhibitor, an antibody to PD-L1, a CTLA-4 inhibitor, a LAG3 inhibitor, a TIM3 inhibitor, a dietary supplement, a VEGF antagonist, surgery, radiation, a chemotherapeutic agent, and a cytotoxic agent.

The present disclosure also provides kits comprising the disclosed targeted immunocytokine (e.g., a fusion protein comprising an immunoglobulin antigen-binding domain of a checkpoint inhibitor and an IL-2 moiety) and PD1 inhibitor (e.g., an anti-PD1 antibody or antigen-binding fragment thereof, such as cemiplimab or a bioequivalent thereof) for therapeutic uses as described herein. Kits typically include a label indicating the intended use of the contents of the kit and instructions for use. As used herein, the term “label” includes any writing, or recorded material supplied on, in or with the kit, or that otherwise accompanies the kit. In some embodiments, the present disclosure provides a kit for treating a subject afflicted with cancer, wherein the kit includes: (a) a therapeutically effective dose of a disclosed targeted immunocytokine; a therapeutically effective dose of a disclosed PD1 inhibitor; and (b) instructions for using the combination of doses in any of the methods disclosed herein.

### Administration Regimens

The present disclosure includes methods that comprise administering to a subject with cancer a combination of the targeted immunocytokine and PD1 inhibitor at a dosing frequency that achieves a therapeutic response. In some embodiments, the targeted immunocytokine and/or the PD1 inhibitor is administered to the subject in one or more doses administered about four times a week, twice a week, once a week, once every two weeks, once every three weeks, once every four weeks, once every five weeks, once every six weeks, once every eight weeks, once every twelve weeks, or less frequently so long as a therapeutic response is achieved.

In some embodiments, when the targeted immunocytokine is administered “before” the PD1 inhibitor, the targeted immunocytokine may be administered more than 12 weeks, about 12 weeks, about 11 weeks, about 10 weeks, about 9 weeks, about 8 weeks, about 7 weeks, about 6 weeks, about 5 weeks, about 4 weeks, about 3 weeks, about 2 weeks, about 1 week, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, or about 30 minutes prior to the administration of the PD1 inhibitor.

In some embodiments, when the targeted immunocytokine is administered “after” the PD1 inhibitor, the targeted immunocytokine may be administered about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, about 1 week, about 2 weeks, about 3 weeks, about 4 weeks, about 5 weeks, about 5 weeks, about 7 weeks, about 8 weeks, about 9 weeks, about 10 weeks, about 11 weeks, about 12 weeks, or more than 12 weeks after the administration of the PD1 inhibitor.

As used herein, “concurrent” administration means that the targeted immunocytokine and PD1 inhibitor are administered to the subject in a single dosage form (e.g., co-formulated) or in separate dosage forms administered to the subject within about 30 minutes or less of each other (i.e., before, after, or at the same time), such as about 15 minutes or less, or about 5 minutes or less. If administered in separate dosage forms, each dosage form may be administered via the same route (e.g., both administered intravenously, subcutaneously, etc.); or, alternatively, each dosage form may be administered via a different route. In any event, administering the components in a single dosage from, in separate dosage forms by the same route, or in separate dosage forms by different routes are all considered “concurrent” administration” for purposes of the present disclosure.

As used herein, “sequential” administration means that each dose of a selected therapy is administered to the subject at a different point in time, e.g., on different days separated by a predetermined interval (e.g., hours, days, weeks, or months). For illustrative purposes, sequential administration may include administering an initial dose of the targeted immunocytokine (or PD1 inhibitor), followed by one or more secondary doses the targeted PD1 inhibitor (or targeted immunocytokine), optionally followed by one or more tertiary doses of the targeted immunocytokine (or PD1 inhibitor). For illustrative purposes, sequential administration may include administering to the subject an initial dose of the targeted immunocytokine (or PD1 inhibitor), followed by one or more secondary doses of the PD1 inhibitor (or targeted immunocytokine), and optionally followed by one or more tertiary doses of the PD1 inhibitor (or targeted immunocytokine).

### Dosage

With respect to pharmaceutical compositions, the disclosed targeted immunocytokine or PD1 inhibitor may each be formulated with one or more carriers, excipients and/or diluents. In some embodiments, the targeted immunocytokine or PD1 inhibitor may be formulated in the form of a fusion protein (e.g., dimeric fusion protein) with one or more carriers, excipients and/or diluents. Pharmaceutical compositions comprising the targeted immunocytokine and/or PD1 inhibitor may be formulated for specific uses, such as for veterinary uses or pharmaceutical uses in humans. The form of the composition (e.g., dry powder, liquid formulation, etc.) and the excipients, diluents and/or carriers used will depend upon the intended therapeutic use and desired mode of administration of the targeted immunocytokine or PD1 inhibitor.

A pharmaceutical composition of the present disclosure may contain either or both of the targeted immunocytokine and PD1 inhibitor. Such pharmaceutical compositions may be administered to a subject by a variety of routes such as orally, transdermally, subcutaneously, intranasally, intravenously, intramuscularly, intratumorally, intrathecally, topically, or locally. In some embodiments, the pharmaceutical composition is administered to the subject intravenously or subcutaneously. Pharmaceutical compositions can be conveniently presented in unit dosage forms containing a predetermined amount of the disclosed targeted immunocytokine and/or PD1 inhibitor per dose.

In general, the amount of targeted immunocytokine and/or PD1 inhibitor administered to a subject according to the methods of the present disclosure is a therapeutically effective amount. As used herein, “therapeutically effective amount” means an amount of the targeted immunocytokine in combination with the PD1 inhibitor that results in one or more of: (a) a reduction in the severity or duration of a symptom of a cancer; (b) enhanced inhibition of tumor growth, or an increase in tumor necrosis, tumor shrinkage and/or tumor disappearance; (c) delay in tumor growth and development; (d) inhibition or retardation or termination of tumor metastasis; (e) prevention of recurrence of tumor growth; (f) increase in survival of a subject with a cancer; and (g) a reduction in the use or need for conventional anti-cancer therapy (e.g., reduced or eliminated use of chemotherapeutic or cytotoxic agents) as compared to an untreated subject or a subject treated with targeted immunocytokine as monotherapy.

In some embodiments, a therapeutically effective amount of the targeted immunocytokine may be from about 0.05 mg to about 600 mg, or from about 0.005 mg/kg to about 10 mg/kg of the subject's body weight. In some embodiments, a therapeutically effective amount of the PD1 inhibitor (e.g., cemiplimab or a bioequivalent thereof) may be from about 0.05 mg to about 600 mg, or from about 0.0001 mg/kg to about 100 mg/kg of the subject's body weight.

### EXAMPLES

The following examples are put forth so as to provide those of ordinary skill in the art with a complete disclosure and description of how to make and use the methods and compositions of the present disclosure and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Anti-PD1-IL2Ra-IL2 Fusion Proteins

Three anti-PD1-IL2Ra-IL2 fusion proteins were generated by expressing a first polynucleotide sequence encoding a heavy chain of an anti-PD1 antibody linked to the N-terminus of a IL2 moiety and a second polynucleotide sequence encoding a light chain of the anti-PD1 antibody in host cells. The IL2 moiety includes IL2 linked to the C-terminus of IL2Ra. The first polynucleotide sequence and the second polynucleotide sequence can be carried on the same or different expression vectors. See US20220402989. Table 1 sets forth the amino acid sequence identifiers of the three anti-PD1-IL2Ra-IL2 fusion proteins. See PCT/US2022/072895.

TABLE 1
Amino acid identifiers of anti-PD1-IL2Ra-IL2 fusion proteins

| | SEQ ID NOs | | | | | | | | | | |
|---|---|---|---|---|---|---|---|---|---|---|---|
| | | | | | | | | | | | IL2 |
| ID | HCVR | HCDR1 | HCDR2 | HCDR3 | LCVR | LCDR1 | LCDR2 | LCDR3 | HC | LC | moiety |
| REGN10595 | 1 | 2 | 3 | 4 | 5 | 6 | 7 | 8 | 9 | 10 | 27 |
| REGN10486 | 11 | 12 | 13 | 14 | 15 | 16 | 7 | 17 | 18 | 19 | 27 |
| REGN10597 | 20 | 21 | 22 | 23 | 5 | 6 | 7 | 8 | 24 | 25 | 27 |

The IL2 moiety (SEQ ID NO: 27) includes an IL2 (SEQ ID NO: 29) linked to the C-terminus of an IL2Ra (SEQ ID NO: 28). The IL2 moiety (SEQ ID NO: 27) is connected to the C-terminus of the heavy chain constant region (SEQ ID NO: 26) of the anti-PD1 antibody via a linker comprising an amino acid sequence of SEQ ID NO: 30.

For REGN10595, the heavy chain (HC) (SEQ ID NO: 9) includes the amino acid sequences of the HCVR (SEQ ID NO: 1), and the heavy chain constant region (SEQ ID NO: 26); and is linked to the IL2 moiety (SEQ ID NO: 27) via a linker (SEQ ID NO: 30).

For REGN10486, the heavy chain (HC) (SEQ ID NO: 18) includes the amino acid sequences of the HCVR (SEQ ID NO: 11)) and the heavy chain constant region (SEQ ID NO: 26), and is linked to the IL2 moiety (SEQ ID NO: 27) via a linker (SEQ ID NO: 30).

For REGN10597, the heavy chain (HC) (SEQ ID NO: 24) includes the amino acid sequences of the HCVR (SEQ ID NO: 20) and the heavy chain constant region (SEQ ID NO: 26), and is linked to the IL2 moiety (SEQ ID NO: 27) via a linker (SEQ ID NO: 30).

Table 2 sets forth the amino acid sequences of the three anti-PD1-IL2Ra-IL2 fusion proteins.

TABLE 2
Amino acid sequences of anti-PD1-IL2Ra-IL2

| SEQ | | |
|---|---|---|
| ID | | |
| NO | SEQUENCE | INFORMATION |
| 1 | QVQLVQSGTEVRKPGSSVKVSCKTSGVTFNNYAITWVRQAPGQGLE | VH; |
| | WMGGIIPVFSPPNYAQKFQGRVTITADESTNTAYMELNSLRSDDTAIYF | REGN10595 |
| | CAREGERGYTYGYDYWGQGTLVTVSS | |
| 2 | GVTFNNYA | HCDR1; |
| | | REGN10595 |
| 3 | IIPVFSPP | HCDR2; |
| | | REGN10595 |
| 4 | AREGERGYTYGYDY | HCDR3; |
| | | REGN10595 |
| 5 | DIQMTQSPSSLSASVGDRVTITCRASQSISSYLNWYQQKPGKAPKLLIY | VL; |
| | AASSLQSGVPSRFSGSGSGTDFTLTISSLQPEDFATYYCQQSYSTPPI | REGN10595 |
| | TFGQGTRLEIK | |
| 6 | QSISSY | LCDR1; |
| | | REGN10595 |
| 7 | AAS | LCDR2; |
| | | REGN10595 |
| 8 | QQSYSTPPIT | LCDR3; |
| | | REGN10595 |
| 9 | QVQLVQSGTEVRKPGSSVKVSCKTSGVTFNNYAITWVRQAPGQGLE | HC; |
| | WMGGIIPVFSPPNYAQKFQGRVTITADESTNTAYMELNSLRSDDTAIYF | REGN10595 |
| | CAREGERGYTYGYDYWGQGTLVTVSSASTKGPSVFPLAPCSRSTSE | |
| | STAALGCLVKDYFPEPVTVSWNSGALTSGVHTFPAVLQSSGLYSLSSV | |
| | VTVPSSSLGTKTYTCNVDHKPSNTKVDKRVESKYGPPCPPCPAPPVA | |
| | GPSVFLFPPKPKDTLMISRTPEVTCVVVDVSQEDPEVQFNWYVDGVE | |
| | VHNAKTKPREEQFNSTYRVVSVLTVLHQDWLNGKEYKCKVSNKGLPS | |
| | SIEKTISKAKGQPREPQVYTLPPSQEEMTKNQVSLTCLVKGFYPSDIAV | |
| | EWESNGQPENNYKTTPPVLDSDGSFFLYSRLTVDKSRWQEGNVFSC | |
| | SVMHEALHNHYTQKSLSLSLGK | |
| 10 | DIQMTQSPSSLSASVGDRVTITCRASQSISSYLNWYQQKPGKAPKLLIY | LC |
| | AASSLQSGVPSRFSGSGSGTDFTLTISSLQPEDFATYYCQQSYSTPPI | REGN10595 |
| | TFGQGTRLEIKRTVAAPSVFIFPPSDEQLKSGTASVVCLLNNFYPREAK | |
| | VQWKVDNALQSGNSQESVTEQDSKDSTYSLSSTLTLSKADYEKHKVY | |
| | ACEVTHQGLSSPVTKSFNRGEC | |
| 11 | EVQLLESGGVLVQPGGSLRLSCAASGFTFSNFGMTWVRQAPGKGLE | VH; |
| | WVSGISGGGRDTYFADSVKGRFTISRDNSKNTLYLQMNSLKGEDTAV | REGN10486 |
| | YYCVKWGNIYFDYWGQGTLVTVSS | |
| 12 | GFTFSNFG | HCDR1; |
| | | REGN10486 |
| 13 | ISGGGRDT | HCDR2; |
| | | REGN10486 |
| 14 | VKWGNIYFDY | HCDR3; |
| | | REGN10486 |
| 15 | DIQMTQSPSSLSASVGDSITITCRASLSINTFLNWYQQKPGKAPNLLIYA | VL; |
| | ASSLHGGVPSRFSGSGSGTDFTLTIRTLQPEDFATYYCQQSSNTPFTF | REGN10486 |
| | GPGTVVDFR | |
| 16 | LSINTF | LCDR1; |
| | | REGN10486 |
| 7 | AAS | LCDR2; |
| | | REGN10486 |
| 17 | QQSSNTPFT | LCDR3; |
| | | REGN10486 |
| 18 | EVQLLESGGVLVQPGGSLRLSCAASGFTFSNFGMTWVRQAPGKGLE | HC; |
| | WVSGISGGGRDTYFADSVKGRFTISRDNSKNTLYLQMNSLKGEDTAV | REGN10486 |
| | YYCVKWGNIYFDYWGQGTLVTVSSASTKGPSVFPLAPCSRSTSESTA | |
| | ALGCLVKDYFPEPVTVSWNSGALTSGVHTFPAVLQSSGLYSLSSVVTV | |
| | PSSSLGTKTYTCNVDHKPSNTKVDKRVESKYGPPCPPCPAPPVAGPS | |
| | VFLFPPKPKDTLMISRTPEVTCVVVDVSQEDPEVQFNWYVDGVEVHN | |
| | AKTKPREEQFNSTYRVVSVLTVLHQDWLNGKEYKCKVSNKGLPSSIEK | |
| | TISKAKGQPREPQVYTLPPSQEEMTKNQVSLTCLVKGFYPSDIAVEWE | |
| | SNGQPENNYKTTPPVLDSDGSFFLYSRLTVDKSRWQEGNVFSCSVM | |
| | HEALHNHYTQKSLSLSLGK | |
| 19 | DIQMTQSPSSLSASVGDSITITCRASLSINTFLNWYQQKPGKAPNLLIYA | LC; |
| | ASSLHGGVPSRFSGSGSGTDFTLTIRTLQPEDFATYYCQQSSNTPFTF | REGN10486 |
| | GPGTVVDFRRTVAAPSVFIFPPSDEQLKSGTASVVCLLNNFYPREAKV | |
| | QWKVDNALQSGNSQESVTEQDSKDSTYSLSSTLTLSKADYEKHKVYA | |
| | CEVTHQGLSSPVTKSFNRGEC | |
| 20 | QVQLVQSGAEVKRPGSSVKVSCKVSGVTFRNFAIIWVRQAPGQGLEW | VH; |
| | MGGIIPFFSAANYAQSFQGRVTITPDESTSTAFMELASLRSEDTAVYYC | REGN10597 |
| | AREGERGHTYGFDYWGQGTLVTVSS | |
| 21 | GVTFRNFA | HCDR1; |
| | | REGN10597 |
| 22 | IIPFFSAA | HCDR2; |
| | | REGN10597 |
| 23 | AREGERGHTYGFDY | HCDR3; |
| | | REGN10597 |
| 5 | DIQMTQSPSSLSASVGDRVTITCRASQSISSYLNWYQQKPGKAPKLLIY | VL; |
| | AASSLQSGVPSRFSGSGSGTDFTLTISSLQPEDFATYYCQQSYSTPPI | REGN10597 |
| | TFGQGTRLEIK | |
| 6 | QSISSY | LCDR1; |
| | | REGN10597 |
| 7 | AAS | LCDR2; |
| | | REGN10597 |
| 8 | QQSYSTPPIT | LCDR3; |
| | | REGN10597 |
| 24 | QVQLVQSGAEVKRPGSSVKVSCKVSGVTFRNFAIIWVRQAPGQGLEW | HC; |
| | MGGIIPFFSAANYAQSFQGRVTITPDESTSTAFMELASLRSEDTAVYYC | REGN10597 |
| | AREGERGHTYGFDYWGQGTLVTVSSASTKGPSVFPLAPCSRSTSEST | |
| | AALGCLVKDYFPEPVTVSWNSGALTSGVHTFPAVLQSSGLYSLSSVVT | |
| | VPSSSLGTKTYTCNVDHKPSNTKVDKRVESKYGPPCPPCPAPPVAGP | |
| | SVFLFPPKPKDTLMISRTPEVTCVVVDVSQEDPEVQFNWYVDGVEVH | |
| | NAKTKPREEQFNSTYRVVSVLTVLHQDWLNGKEYKCKVSNKGLPSSI | |
| | EKTISKAKGQPREPQVYTLPPSQEEMTKNQVSLTCLVKGFYPSDIAVE | |
| | WESNGQPENNYKTTPPVLDSDGSFFLYSRLTVDKSRWQEGNVFSCS | |
| | VMHEALHNHYTQKSLSLSLGK | |
| 25 | DIQMTQSPSSLSASVGDRVTITCRASQSISSYLNWYQQKPGKAPKLLIY | LC; |
| | AASSLQSGVPSRFSGSGSGTDFTLTISSLQPEDFATYYCQQSYSTPPI | REGN10597 |
| | TFGQGTRLEIKRTVAAPSVFIFPPSDEQLKSGTASVVCLLNNFYPREAK | |
| | VQWKVDNALQSGNSQESVTEQDSKDSTYSLSSTLTLSKADYEKHKVY | |
| | ACEVTHQGLSSPVTKSFNRGEC | |
| 26 | ASTKGPSVFPLAPCSRSTSESTAALGCLVKDYFPEPVTVSWNSGALTS | Heavy chain |
| | GVHTFPAVLQSSGLYSLSSVVTVPSSSLGTKTYTCNVDHKPSNTKVDK | constant region |
| | RVESKYGPPCPPCPAPPVAGPSVFLFPPKPKDTLMISRTPEVTCVVVD | |
| | VSQEDPEVQFNWYVDGVEVHNAKTKPREEQFNSTYRVVSVLTVLHQ | |
| | DWLNGKEYKCKVSNKGLPSSIEKTISKAKGQPREPQVYTLPPSQEEMT | |
| | KNQVSLTCLVKGFYPSDIAVEWESNGQPENNYKTTPPVLDSDGSFFLY | |
| | SRLTVDKSRWQEGNVFSCSVMHEALHNHYTQKSLSLSLGK | |
| 27 | ELCDDDPPEIPHATFKAMAYKEGTMLNCECKRGFRRIKSGSLYMLCTG | IL2 moiety |
| | NSSHSSWDNQCQCTSSATRNTTKQVTPQPEEQKERKTTEMQSPMQ | (IL2Ra + IL2) |
| | PVDQASLPGHCREPPPWENEATERIYHFVVGQMVYYQCVQGYRALH | |
| | RGPAESVCKMTHGKTRWTQPQLICTGGGGGSGGGGSGGGGSGGG | |
| | GSGGGGSAPTSSSTKKTQLQLEHLLLDLQMILNGINNYKNPKLTRMLT | |
| | FKFYMPKKATELKHLQCLEEELKPLEEVLNLAQSKNFHLRPRDLISNIN | |
| | VIVLELKGSETTFMCEYADETATIVEFLNRWITFCQSIISTLT | |
| 28 | ELCDDDPPEIPHATFKAMAYKEGTMLNCECKRGFRRIKSGSLYMLCTG | hIL2Ra |
| | NSSHSSWDNQCQCTSSATRNTTKQVTPQPEEQKERKTTEMQSPMQ | |
| | PVDQASLPGHCREPPPWENEATERIYHFVVGQMVYYQCVQGYRALH | |
| | RGPAESVCKMTHGKTRWTQPQLICTG | |
| 29 | APTSSSTKKTQLQLEHLLLDLQMILNGINNYKNPKLTRMLTFKFYMPKK | hIL-2 |
| | ATELKHLQCLEEELKPLEEVLNLAQSKNFHLRPRDLISNINVIVLELKGS | |
| | ETTFMCEYADETATIVEFLNRWITFCQSIISTLT | |
| 30 | GGGGSGGGGSGGGGS | Linker |
| 31 | GGGGSGGGGSGGGGSGGGGSGGGGS | Linker |
| 32 | GGGGS | Linker |

### Example 2: A PD1-Targeted, Receptor-Masked IL-2 Immunocytokine that Engages IL-2Ra Strengthens T Cell-Mediated Anti-Tumor Therapies

This example relates to a PD1-targeted, receptor-masked IL-2 immunocytokine that engages IL-2Ra. Interleukin-2 (IL-2 or IL2), a key cytokine for T cell proliferation, differentiation, and effector function, has been used for the treatment of metastatic melanoma and renal cell carcinoma and induced complete, durable tumor regression in some patients. However, its broader use in cancer immunotherapy has been limited by severe toxicity. A new generation of IL-2 therapies with decreased binding to IL-2 receptor alpha chain (IL-2Ra) to mitigate toxicity and Treg expansion has had limited clinical success.

Here, it is demonstrated that the ability to engage IL-2Ra is critical for the anti-tumor activity of a systemic IL-2 therapy. Despite inducing robust expansion of CD8+ T cells and NK cells over Tregs in the circulation, an IL-2 mutein with abolished IL-2Ra binding has limited anti-tumor efficacy compared to wild-type IL-2. Based on these findings, a PD1-targeted, receptor-masked IL-2 immunocytokine was developed with attenuated systemic IL-2 activity but maintained capacity to engage IL-2Ra on PD1+ T cells. PD1-IL2Ra-IL2 (REGN10597) shows PD1-targeting-dependent IL-2 activity in vitro and drives selective expansion of tumor-infiltrating PD1+ CD8+ T cells with vigorous effector profiles in vivo. PD1-IL2Ra-IL2 treated mice displayed no signs of systemic toxicities observed with unmasked IL-2 treatment, with robust tumor growth control and enhanced survival. Finally, it is demonstrated that PD1-IL2Ra-IL2 can be effectively combined with another T cell-mediated immunotherapy, PD1 inhibitors, to potentiate antitumor responses. Collectively, these results provide unique insights into the functional mechanism of IL-2 based therapeutics and highlight the therapeutic potential of PD1-IL2Ra-IL2 as a novel targeted, receptor masked, and “alpha-maintained” IL-2 therapy for cancer treatment.

The clinical success of immune checkpoint inhibitors, as well as the clear potential of several emerging modalities such as cell therapies and bispecific T cell-engagers, have highlighted the promise of cancer immunotherapies and transformed cancer treatments (Bagchi (2021), Kawalekar (2016), van de Donk (2023)). T cells are major drivers of anti-cancer activity primarily through antigen-directed cytotoxicity against tumor cells (Waldman (2020)). Although checkpoint inhibitors have improved patient outcomes across several cancer types, the majority of patients still fail to achieve durable responses. By itself, removing these physiologic brakes is insufficient in patients whose antitumor T-cell immunity is actively curtailed by other barriers such as poor cytotoxic T cell infiltration and function in the immunosuppressive tumor microenvironment (Bagchi (2021), Fridman (2012)).

Cytokines are secreted proteins that can act in an autocrine and paracrine manner to activate and tune immune responses. Their immunomodulatory activities have provided a strong rationale for the exploration of cytokine-based therapeutics for cancer treatment (Waldmann (2018)). IL-2 is a central T cell cytokine that promotes T cell proliferation, differentiation and effector function. It can signal through two types of receptor complexes formed from three subunits, IL-2Rα (IL-2a, CD25), IL-2Rβ (IL2Rb, CD122), and IL-2Rγ (IL-2Rb, CD132). The heterodimeric IL-2Rβ/γ (IL-2Rb/g) receptor binds to IL-2 with intermediate affinity and is mostly expressed on resting CD8+ T cells and NK cells; the heterotrimeric IL-2Rα/β/γ (IL-2Ra/b/g) receptor binds to IL-2 with high affinity and is primarily expressed on Tregs and activated T cells (Spolski (2018)). Recombinant IL-2 (aldesleukin) has been approved for the treatment of advanced melanoma and renal cell carcinoma and induced complete, durable tumor regression in some patients (Rosenberg (2014)). In the clinical setting, IL-2 requires high doses and frequent administration to overcome its short in vivo half-life and effectively activate effector T cells. However, severe systemic toxicities associated with high-dose IL-2 therapy have limited its practical use (Dutcher (2014), Vial (1992). To reduce toxicities and limit Treg expansion associated with high-dose IL-2 treatment, a new generation of IL-2 therapies with decreased binding to IL-2Ra, and therefore biased activity towards IL-2Rb/g dimeric receptor to improve stimulation of CD8+ T and NK cells, have been developed (Charych (2016), Klein (2017), Lopes (2020), Ptacin (2021)). While some of these molecules seem to deliver an improved safety profile in clinical trials, limited single agent efficacy has been reported from these studies (Bentebibel (2019), Janku (2021)).

Given the pleiotropic function of IL-2 and the broad expression of its receptors on different cell types, attempts have been made to selectively deliver IL-2 to the tumor microenvironment via fusing IL-2 to antibodies targeting tumor-associated antigens or tumor stroma. However, such strategies have been less efficient than expected in localizing IL-2 activity to the tumor tissues (Tzeng (2015), van Brummelen (2018), Waldhauer (2021)). An alternative approach to improve efficacy and circumvent systemic toxicity of IL-2 is to deliver IL-2 signaling specifically to T cells that are tumor-reactive. One cell-surface marker for such tumor-reactive T cells is the coinhibitory receptor programmed cell death protein-1 (PD1) (Gros (2014), Gros (2016)). PD1 is expressed on the surface of antigen-activated T cells and antibodies that bind to PD1 may enable targeted delivery of IL-2. In this regard, PD1-targeted IL-2Ra-attenuated IL-2 mutein molecules have been recently developed to provide IL-2R agonism preferentially to PD1+ tumor-reactive T cells (Codarri Deak (2022), Ren (2022)).

Herein, it is shown that engagement of IL-2Ra is critical for the anti-tumor activity of a systemic IL-2 therapy. Despite being more efficient in expanding circulating CD8+ T cells and NK cells over Tregs, an IL-2 mutein with abolished IL-2Ra has diminished anti-tumor efficacy when compared to wild-type IL-2. A PD1-targeted, receptor masked IL-2 immunocytokine was further developed, termed PD1-IL2Ra-IL2 (REGN10597). Receptor masking results in attenuated IL-2 activity for both IL-2Ra/b/g trimeric and IL-2Rb/g dimeric receptors, while PD1-targeting selectively reconstitutes IL-2Ra/b/g agonism on PD1+ tumor-reactive T cells as PD1-IL2Ra-IL2 consistently demonstrated PD1-targeting-dependent IL-2 activity in vitro driving selective expansion of tumor-infiltrating PD1+CD8 T cells with highly functional effector profiles in vivo. Treatment with PD1-IL2Ra-IL2 results in robust tumor growth inhibition and enhanced survival in mice. Finally, it was demonstrated that PD1-IL2Ra-IL2 can be effectively combined with another T cell-mediated immunotherapy: anti-PD1 antibody, to augment antitumor responses. Collectively, these results shed light on the functional mechanism of IL-2 based therapeutics, highlight the therapeutic potential of PD1-IL2Ra-IL2, and inform its clinical development both as a monotherapy and in combination with other immunotherapies for the treatment of cancer.

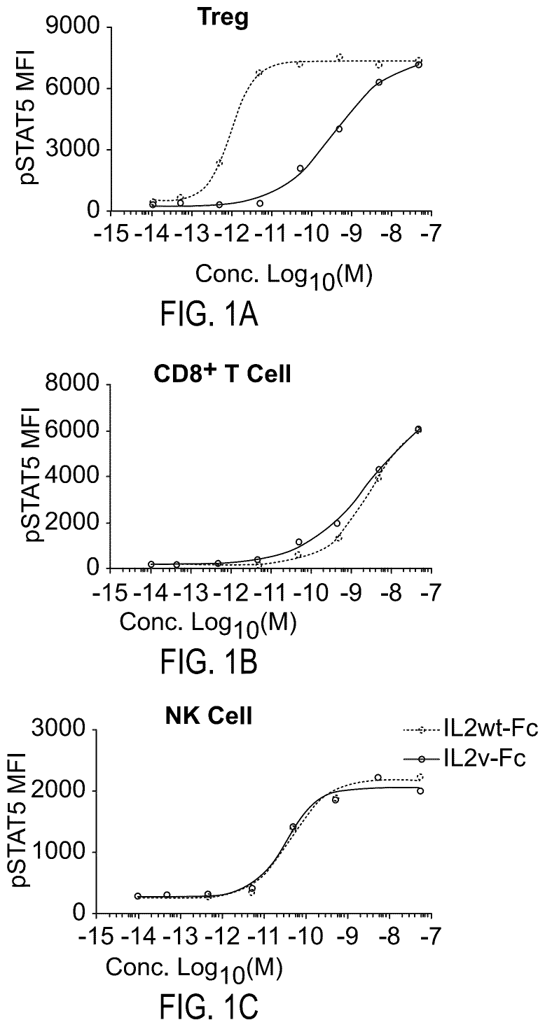

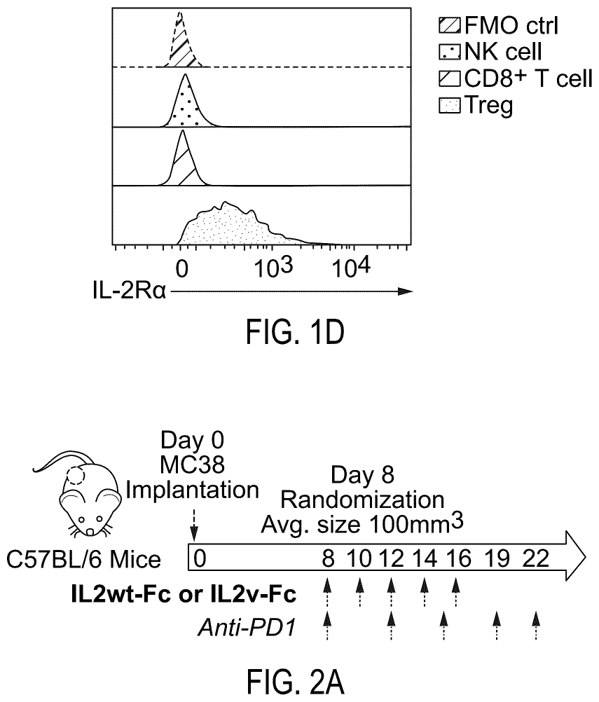

Requirement of IL-2Ra engagement for anti-tumor activity of systemic IL-2: With the intent of reducing systemic IL-2 toxicity and selectively expanding IL-2Rb/g-expressing CD8+ T cells and NK cells over IL-2Ra/b/g-expressing endothelial cells and Tregs, the majority of IL-2-based cancer therapeutics currently being evaluated in clinical trials utilize an IL-2Rb/g-biased IL-2 strategy (Raeber (2022)). In these approaches, however, the requirement of IL-2Ra in driving anti-tumor activity remains to be defined. To understand the requirement of IL-2Ra engagement in IL-2 mediated anti-tumor activity, the in vitro and in vivo activities were compared of wild-type (wt) IL-2 or and an IL-2Rb/g-biased IL-2 variant (IL2v) that carries three mutations to specifically abolish binding to IL-2Ra (Klein (2017)). Both molecules were generated in an Fc fusion format to extend half-life. Human PBMCs were stimulated with a titration of IL2 wt-Fc or IL2v-Fc for 20 mins. As expected, when IL2 wt-Fc and IL2v-Fc were compared for their ability to induce IL-2R signaling on primary human lymphocytes, IL2v-Fc showed reduced potency in inducing STAT5 phosphorylation only on IL-2Ra⁺ Tregs, but not on CD8⁺ T cells or NK cells that are IL-2Ra (FIGS. 1A-1D). Consistently, while administration with IL2 wt-Fc induced preferential expansion of Tregs over CD8⁺ T cells in the spleen of mice, IL2v-Fc treatment led to robust systemic expansion of CD8⁺ T cells and NK cells over Tregs. Data are representative of three independent experiments.

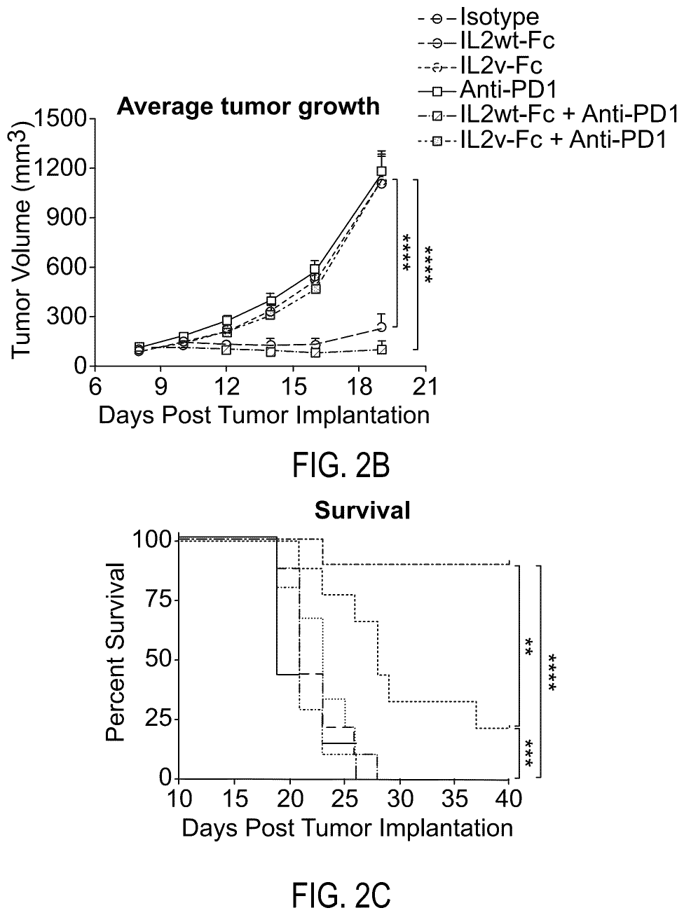

The anti-tumor efficacy of IL2 wt-Fc and IL2v-Fc were compared, both as single agents and in combination with anti-PD1 blockade in the syngeneic MC38 colon carcinoma model (FIGS. 2A-2C). C57BL/6 mice bearing subcutaneously established MC38 tumors (100 mm³) were randomized (10 mice per group), and treated intraperitoneally with isotype (10 mg/kg), IL2 wt-Fc (0.75 mg/kg), IL2v-Fc (0.75 mg/kg), anti-mPD1 (10 mg/kg), IL2 wt-Fc (0.75 mg/kg)+anti-mPD1 (10 mg/kg), or IL2v-Fc (0.75 mg/kg)+anti-mPD1 (10 mg/kg) on indicated days. measured. Statistical significance for average tumor growth (mean+SEM) was determined by two-way ANOVA and Bonferroni's multiple comparisons tests (****P≤0.0001). Statistical significance in survival curves was determined by Kaplan-Meier analysis with the log-rank test (**P≤0.01, ***P≤0.001, ****P≤0.0001). Data are representative of three independent experiments.

Surprisingly, IL2v-Fc showed minimal effect on tumor growth and mouse survival, whereas IL2 wt-Fc led to significantly suppressed tumor growth and extended survival. Combination with anti-PD1 blockade further enhanced the efficacy of IL2 wt-Fc resulting in more mice achieving complete tumor regression, but had little improvement on the anti-tumor activity of IL2v-Fc (FIGS. 2B and 2C). The observation that IL2v-Fc induced robust expansion of splenic CD8⁺ T cells and NK cells but had limited anti-tumor efficacy indicated that the systemically expanded CD8⁺ T cells by IL2v-Fc may be largely irrelevant to anti-tumor immunity. To test if the same immune profiles are induced in the tumor site, MC38-tumor bearing mice were treated with IL2 wt-Fc versus IL2v-Fc, and tumor-infiltrating lymphocytes were analyzed by flow-cytometry. In sharp contrast to what was observed in the spleen and blood from the same mice, IL2 wt-Fc but not IL2v-Fc treatment led to robust expansion of CD8⁺ T cells in the tumor. Neither IL2 wt-Fc nor IL2v-Fc expanded Tregs in the tumor, while IL2v-Fc but not IL2 wt-Fc increased NK cells in the tumor. Distinct from CD8⁺ T cells in the spleen and blood, tumor infiltrating CD8⁺ T cells have upregulated expression of both IL-2Ra and PD1, suggesting they are actively stimulated by antigens in the tumor environment-likely representing tumor-reactive T cells (Kalia (2010)). The selective expression of IL-2Ra on CD8⁺ T cells in the tumor also provides an explanation of why these cells are more effectively stimulated by IL2 wt-Fc over IL2v-Fc. Collectively, these results suggest that although IL-2Rb/g-biased IL-2 can lead to systemic expansion of CD8⁺ T cells and NK cells, the expanded cells are not tumor-specific. Therefore, the ability to engage IL-2Ra is important in IL-2 therapy design to effectively stimulate tumor-reactive T cells and control tumor growth.

Engineering and characterization of a PD1-targeted, receptor-masked IL-2: Based on this finding, a novel IL-2 therapeutic was developed that: (1) maintains ability to engage IL-2Ra to maximize anti-tumor potential; (2) has attenuated IL-2 activity to avoid nonspecific activation of irrelevant cells and systemic toxicity; (3) includes targeted delivery to selectively activate tumor-reactive T cells.

The use of ligand-binding domains of IL-2Ra and IL-2Rb, which bind IL-2 with relatively higher affinity among all three IL-2R subunits, was explored as a possible way to mask and attenuate IL-2 (Myszka (1996)). The wild-type sequence for IL-2 was used so that the unmasked active form retains the ability to engage endogenous IL-2Ra. All constructs were generated in either N- or C-terminal Fc fusion format with different relative orders between IL-2 and the receptor mask. Compared to unmasked controls, different receptor-masked formats led to various degrees of attenuation in IL-2 activity on YT/STAT5-Luciferase reporter cell lines engineered to either knock out or to overexpress IL-2Ra, therefore representing IL-2Rb/g+ or IL-2Ra/b/g+ cell lines respectively. Along with unmasked IL-2 controls, all masked IL-2 molecules showed higher activity in the presence of IL-2Ra, suggesting retained ability to bind IL-2Ra on cells. Based on attenuation as well as protein expression and aggregation profiles of each molecule, the Fc-IL2Ra-IL2 format was selected as a lead “masked” IL2 format. Further efforts to optimize the lengths of the two flexible glycine-serine (GGGGS)ₙ linkers in Fc-IL2Ra-IL2, one connecting Fc and IL2Ra and the other connecting IL2Ra and IL2, suggested little impact of the length of either linker on the degree of attenuation in IL-2 activity. The Fc-(GGGGS)₃-IL2Ra-(GGGGS)₅-IL2 construct was selected for use with an antibody targeting domain. PD1 has been used as a marker enriched on tumor-reactive T cells (Gros (2014)). Additionally, expression of PD1 by virtually all expanded intratumoral CD8+ T cells following treatment with IL2 wt-Fc also suggests PD1 can be a good target for delivering masked IL-2 to tumor reactive T cells. The IL2Ra-IL2 moiety was therefore attached to the C-terminus of an anti-PD1 antibody (hereinafter termed PD1-IL2Ra-IL2, or REGN10597) to selectively reconstitute IL-2 activity on PD1+ tumor-reactive T cells. PD1-IL2Ra-IL2 is based on a hinge-stabilized, effector function-minimized IgG4 isotype as previously described (Davis (2016), Skokos (2020)).

The configuration of masking in PD1-IL2Ra-IL2 was characterized, taking advantage of the unique dynamic half-molecule exchange (also known as “Fab-arm exchange”) behavior of IgG4 antibodies (van der Neut Kolfschoten, 2007). Using native mass spectrometry analysis, the ability of PD1-IL2Ra-IL2 to undergo half-molecule exchange with another IgG4 antibody when disulfide bonds between heavy chains were removed by limited reduction was examined. In contrast to an unmasked PD1-IL2 control molecule which displayed an apparent peak of half-molecule exchanged product, no half-molecule exchange product was observed when PD1-IL2Ra-IL2 was mixed with another IgG4 antibody. This result indicates that PD1-IL2Ra-IL2 predominantly adopts a trans-sequestered conformation, where inter-chain interaction between the IL2Ra mask and the IL2 cytokine prevents half-molecule exchange. Interestingly, a similar preference for intermolecular interaction was previously reported with a mouse IL2-IL2Ra fusion protein which by itself forms a “head-to-tail” dimer, despite the absence of Fc-mediated dimerization and the reverse order in which the cytokine and the receptor are fused to each other (Ward, 2018).

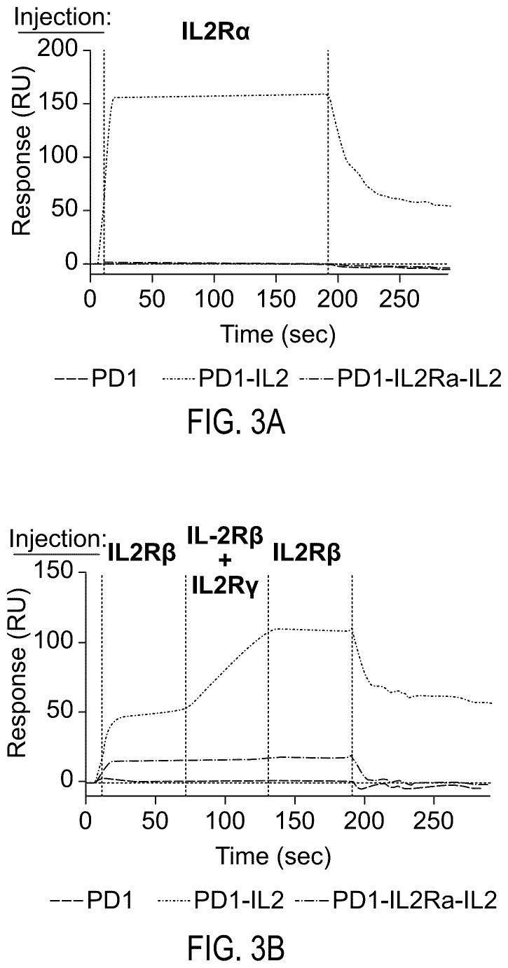

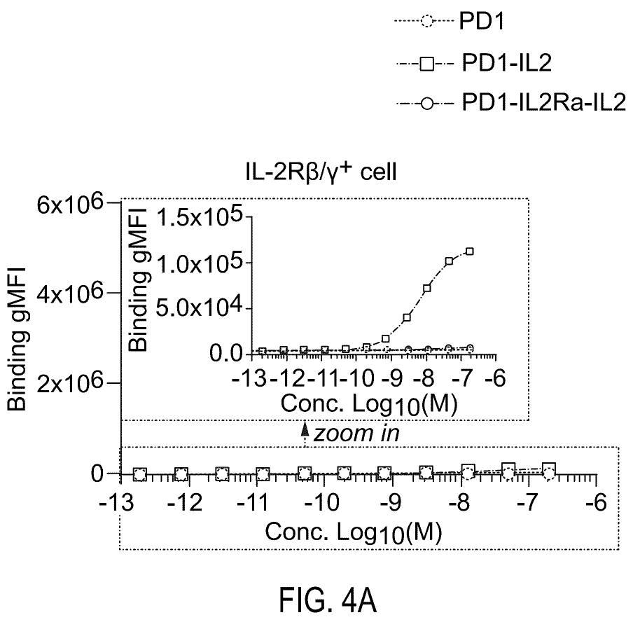

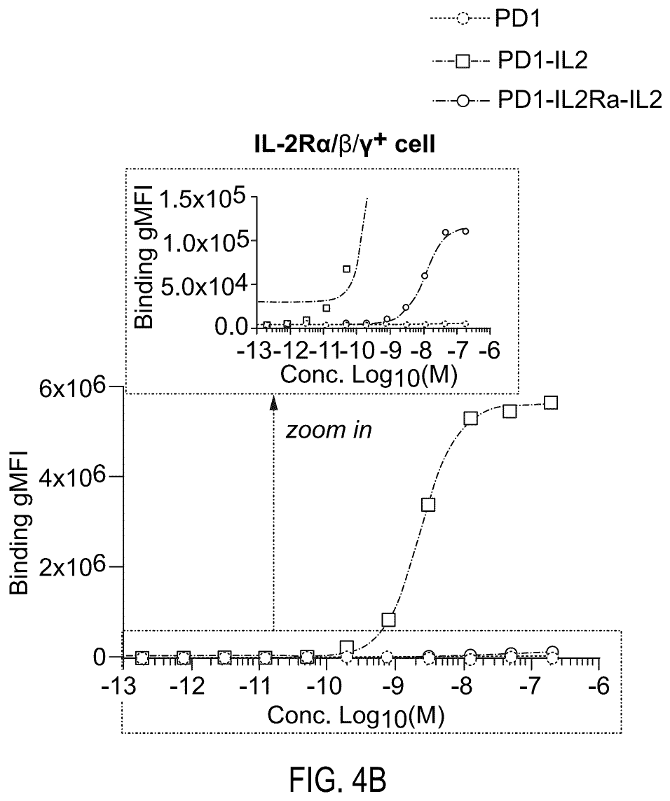

PD1-IL2Ra-IL2, the parental PD1 Ab, or PD1-IL2 were captured by surface-coupled anti-hFab, followed by subsequent injections of indicated IL-2 receptor subunit proteins Surface Plasmon Resonance (SPR) measurements at 25° C. and pH 7.4. In comparison with an unmasked PD1-IL2 control, trans-masking in PD1-IL2Ra-IL2 resulted in a reduced percentage of active IL-2 capable of binding to each IL-2 receptor subunit, IL-2Ra, IL-2Rb, and IL-2Rg (FIGS. 3A and 3B). To examine if masking prevents PD1-IL2Ra-IL2 interaction with cell surface IL-2 receptors, the above-mentioned IL-2Rb/g+ or IL-2Ra/b/g+ YT reporter cell lines, further engineered to knockout PD1 were used. In comparison to the unmasked PD1-IL2 control, PD1-IL2Ra-IL2 demonstrated greatly reduced binding signal to both cell lines. As with PD1-IL2, PD1-IL2Ra-IL2 showed stronger binding to IL-2Ra/b/g+ cell than to IL-2Rb/g+ cell, indicating enhanced binding in the presence of IL-2Ra (FIGS. 4A-4B). Collectively, these results suggest that the vast majority of IL-2 in PD1-IL2Ra-IL2 is trans-masked and inactive, while infrequent unmasking releases fully active IL-2 with retained ability to engage IL-2Ra.

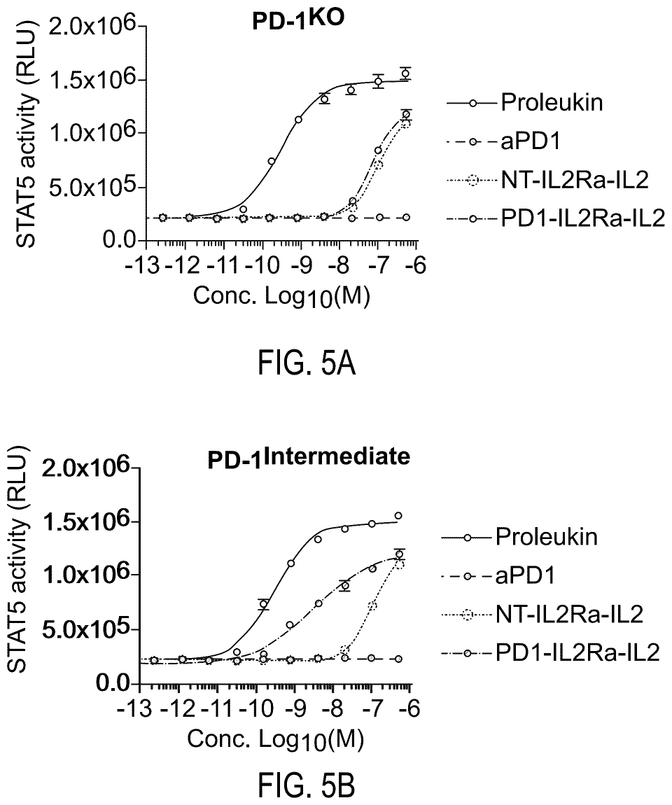

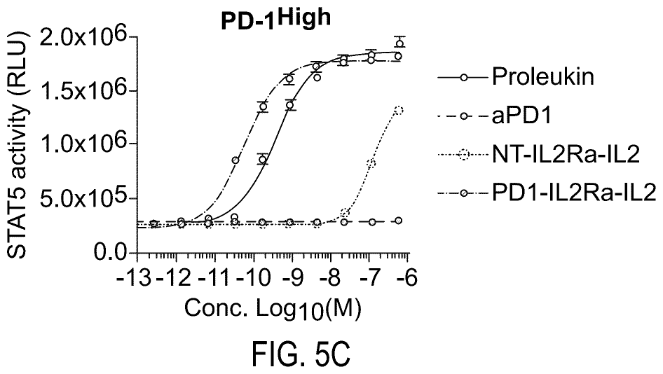

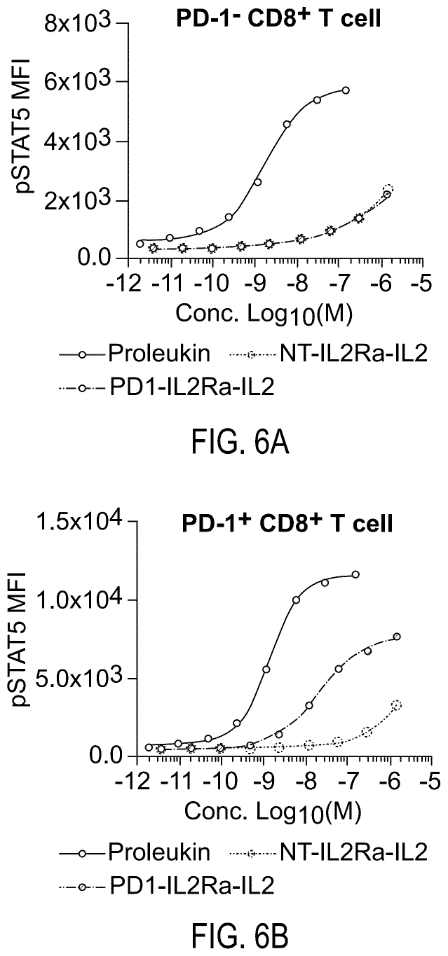

PD1-targeting enhances the activity of receptor masked IL-2: To assess the role of PD1 targeting for activity of the disclosed receptor masked IL-2, IL-2 agonist activity of PD1-IL2Ra-IL2 was compared to an isotype matched non-targeting NT-IL2Ra-IL2 (an anti-human MUC16-IL2RA-IL2 fusion control) using IL-2Rb/g+ or IL-2Ra/b/g+ YT/STAT5-Luc reporter cell lines. On cell lines engineered to lack PD-1 expression, both PD1-IL2Ra-IL2 and NT-IL2Ra-IL2 showed lower potency than recombinant human IL-2 (rhIL-2, Proleukin) in triggering STAT5 activation, consistent with mask-mediated reduced binding (FIGS. 5A-5C). Increased PD1 expression on both IL-2Rβb/g+ and IL-2Ra/b/g+ YT/STAT5-Luc cells drove enhanced STAT5 activation by PD1-IL2Ra-IL2, whereas the potency of NT-IL2Ra-IL2 was not affected by the presence of PD1. Each data point is shown as mean±SD of three technical replicates in the same experiment. Data are representative of at least three experiments. To assess their ability to trigger IL-2R activation-mediated STAT5 phosphorylation on primary human T cells, PD1-IL2Ra-IL2 and NT-IL2Ra-IL2 were incubated with PBMCs. Human PBMCs were stimulated with a titration of each indicated molecule for 20 mins. Data are representative of three independent experiments performed on different donors. While both exhibited equally attenuated potency relative to rhIL-2 on PD1− CD8+ T cells, PD1-IL2Ra-IL2 showed enhanced potency over NT-IL2Ra-IL2 on PD1+CD8+ T cells (FIGS. 6A-6B). Together, PD1-IL2Ra-IL2 demonstrated PD1-targeting enhanced IL-2R agonism in assays performed on both engineered cell lines and primary human T cells.

To further test whether PD1-targeting improves the ability of masked IL-2 to promote T cell activation, a mixed lymphocyte reaction (MLR) assay where T cells from one donor was stimulated with growth arrested allogeneic PBMCs from a different donor in the presence of increasing amounts of PD1-IL2Ra-IL2 or other control molecules was performed. Compared to NT-IL2Ra-IL2, PD1-IL2Ra-IL2 more potently promotes T cell activation measured by production of effector cytokines TNFα and IFNγ from cells stimulated with allogeneic PBMCs.

PD1-IL2Ra-IL2 shows improved specificity and reduced toxicity in vivo: Given its promising masking and PD1-targeting profile in vitro, it was examined if these elements of PD1-IL2Ra-IL2 could enhance specificity and safety of IL-2 administration in vivo. To assess the specificity of PD1-IL2Ra-IL2 in vivo, the expansion of PD1+CD8+ T cells versus PD1− CD8+ T cells in the blood of treated human PD1 knock-in mice was examined. Human PD1 knock-in mice (n=6 per group) bearing established MC38 tumors were treated twice with 0.5 mg/kg of each indicated molecule via intraperitoneal injection on Days 0 and 3. On Day 6, peripheral blood leukocytes were analyzed by flow cytometry for PD1−CD8+ T cell and PD1+CD8+ T cell counts per ml of blood. Results are presented as means±SD. Statistical analyses were performed using one-way ANOVA with Tukey's multiple comparisons tests (**P≤0.01, ***P≤0.001). Data are representative of two experiments.

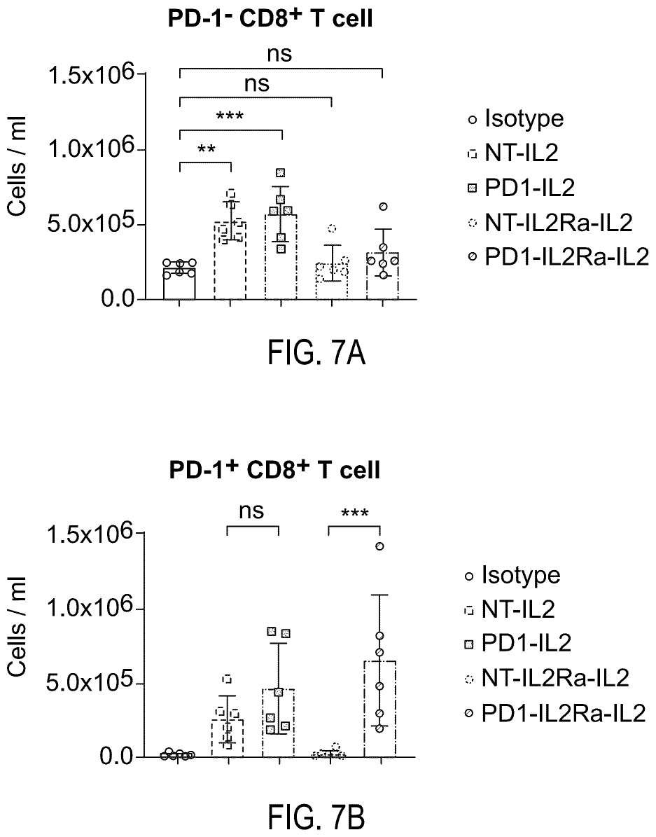

In the absence of masking, NT-IL2 and PD1-IL2 led to expansions of both PD1- and PD1+CD8+ T cells, showing no selectivity. PD1-targeting in PD1-IL2 did not improve selectivity on PD1+ cells (FIG. 7A). In the presence of masking, NT-IL2Ra-IL2 expanded neither PD1− or PD1+CD8+ T cells, whereas PD1-IL2Ra-IL2 led to a selective expansion of PD1+CD8+ T cells with a minimal effect on PD1− CD8+ T cells (FIG. 7B). These results suggest that while masking attenuates systemic activity of IL-2, PD1 targeting allows selective reconstitution of activity on PD1+ cells, both of which are important for specificity in vivo.

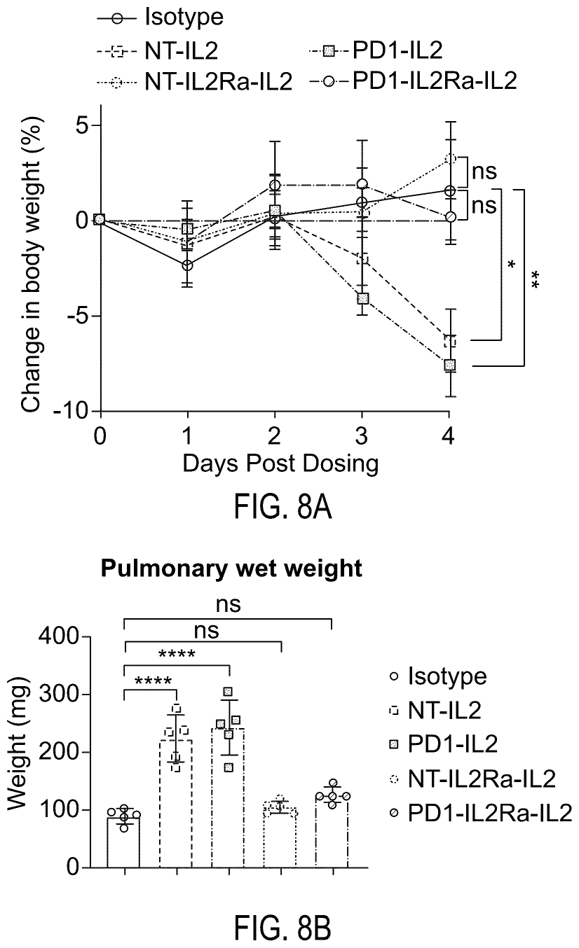

One major dose-limiting side effect associated with systemic IL-2 therapy is pulmonary edema (Vial (1992)). To assess this issue with PD1-IL2Ra-IL2, human PD-1 knock-in mice (n=5 per group) were treated daily with isotype control (1.2 mg/kg), NT-IL2 (1.5 mg/kg), PD1-IL2 (1.5 mg/kg), NT-IL2Ra-IL2 (1.8 mg/kg), or PD1-IL2Ra-IL2 (1.8 mg/kg) via intraperitoneal injection for a total of 4 injections. Dose levels represent equimolar amounts of each agent. Body weight changes were measured every day throughout the experiment. Statistical analyses were performed using two-way ANOVA with Bonferroni's multiple comparisons tests (*P≤0.05, **P≤ 0.01). One day after the last dose, mice were euthanized, and pulmonary wet weight was determined by subtracting dry lung weight (after desiccation at 55° C. for 72 hrs) from fresh lung weight (immediately after collection). Statistical analyses were performed using one-way ANOVA with Dunnett's multiple comparisons tests (****P≤0.0001). Data are representative of two experiments. Daily administration of unmasked NT-IL2 or PD1-IL2 led to significant body weight loss and pulmonary edema observed as increases in pulmonary wet weight. In contrast, daily injection of the same molar amounts of NT-IL2Ra-IL2 or PD1-IL2Ra-IL2 did not result in changes to body weight or wet pulmonary weight, suggesting diminished systemic IL-2 toxicity in the presence of masking (FIGS. 8A-8B). These data demonstrate that PD1-IL2Ra-IL2 has enhanced specificity and reduced toxicity compared to unmasked IL2 in vivo.

PD1-IL2Ra-IL2 exerts potent antitumor activity in syngeneic tumor models: The anti-tumor efficacy of PD1-IL2Ra-IL2 was assessed in several syngeneic mouse tumor models, including B16F10 melanoma, MCA205 fibrosarcoma, TRAMP-C2 prostate cancer, as well as Colon 26 and MC38 colorectal cancers. Due to lack of binding of the anti-huPD1 targeting antibody to mouse PD1, tumor-bearing mice were administered with either the anti-huPD1-IL2Ra-IL2 molecule (REGN10597) to human PD1 knock-in mice or a mouse surrogate anti-mPD1-IL2Ra-IL2 molecule to wild-type C57BL/6 or BALB/c mice. Human PD-1 knock-in mice (8 mice per group) were implanted subcutaneously with B16F10 or MCA205 tumors. C57BL/6 mice (6-7 mice per group) were implanted subcutaneously with TRAMP-C2 tumor cells. BALB/c mice (6 mice per group) were implanted subcutaneously with Colon26 tumor cells. When tumors were established, mice were randomized into groups and treated with indicated molecules and doses on the indicated days via intraperitoneal injection. Tumor growth (mean±SEM) was monitored over time and the number of tumor free (TF) mice at the end of the study is indicated for select tumor models.

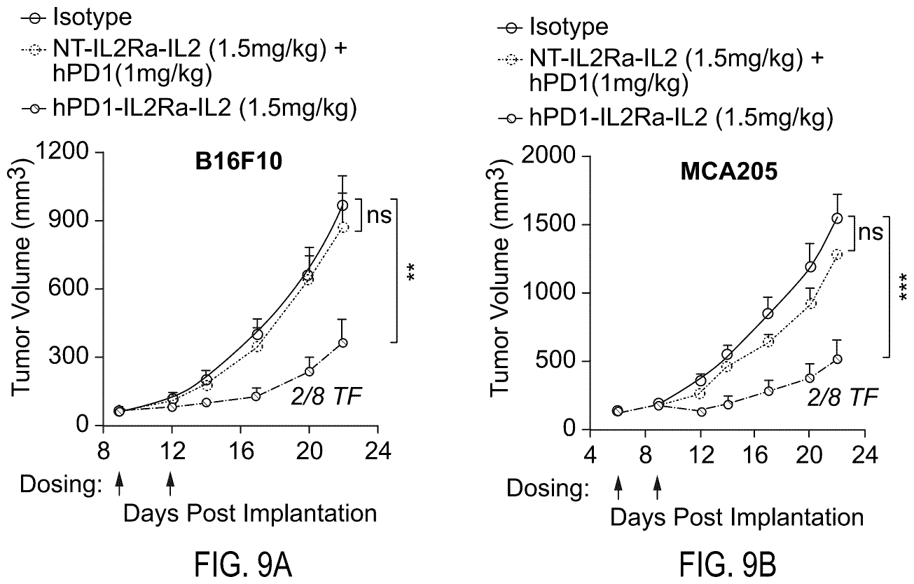

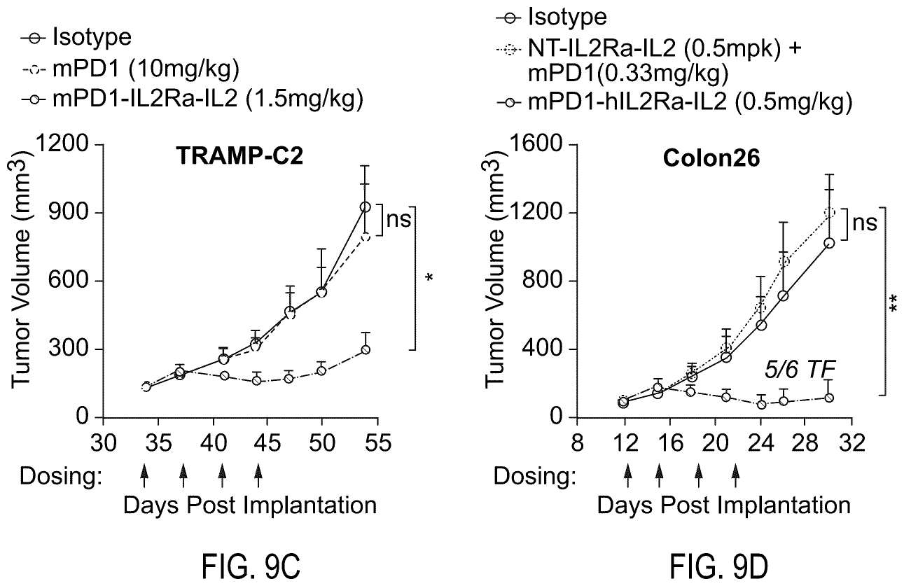

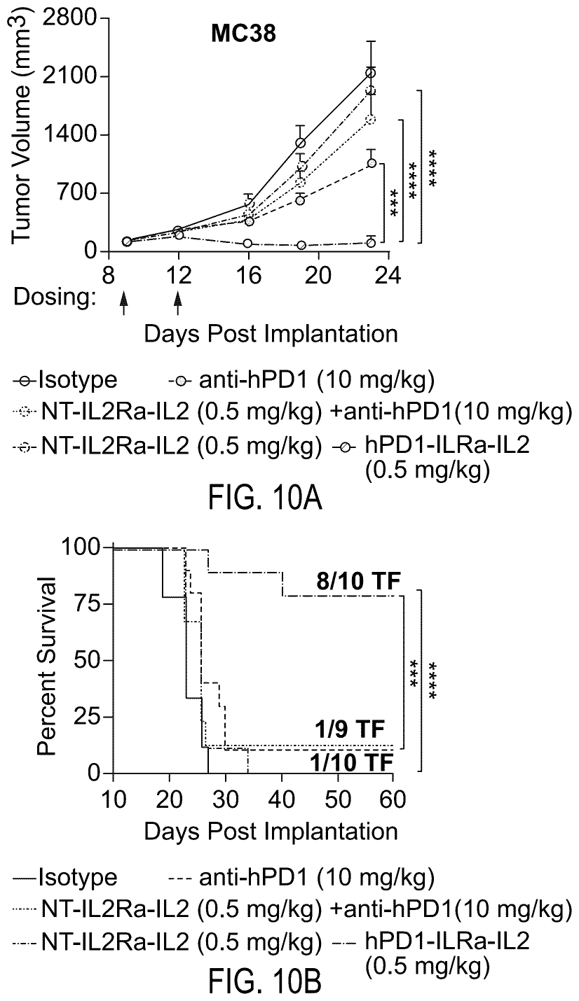

Robust anti-tumor activity of PD1-IL2Ra-IL2 was observed in all the models tested (FIGS. 9A-9D and FIGS. 10A-10B). For the MC38 model experiment, human PD-1 knock-in mice bearing subcutaneously established MC38 tumors (120 mm³) were randomized (9-10 mice per group) and then treated with indicated molecules and doses on the indicated days via intraperitoneal injection. Tumor growth (mean±SEM) (FIG. 10A) and mice survival (FIG. 10B) were monitored over time, and the number of TF mice at the end of the study is indicated for select groups. Data are representative of at least three experiments. Statistical analyses for tumor growth were performed using two-way ANOVA with Bonferroni's multiple comparisons tests (*P≤0.05, **P≤0.01, ***P≤0.001, ****P≤0.0001). All statistical significance for mice survival were determined by Kaplan-Meier analyses with the log-rank tests (***P≤0.001, ****P≤0.0001). The potency of PD1-IL2Ra-IL2 in inhibiting tumor growth and improving tumor-free survival was far superior over NT-IL2Ra-IL2, an anti-PD1 blocker, or their combination (FIG. 10).

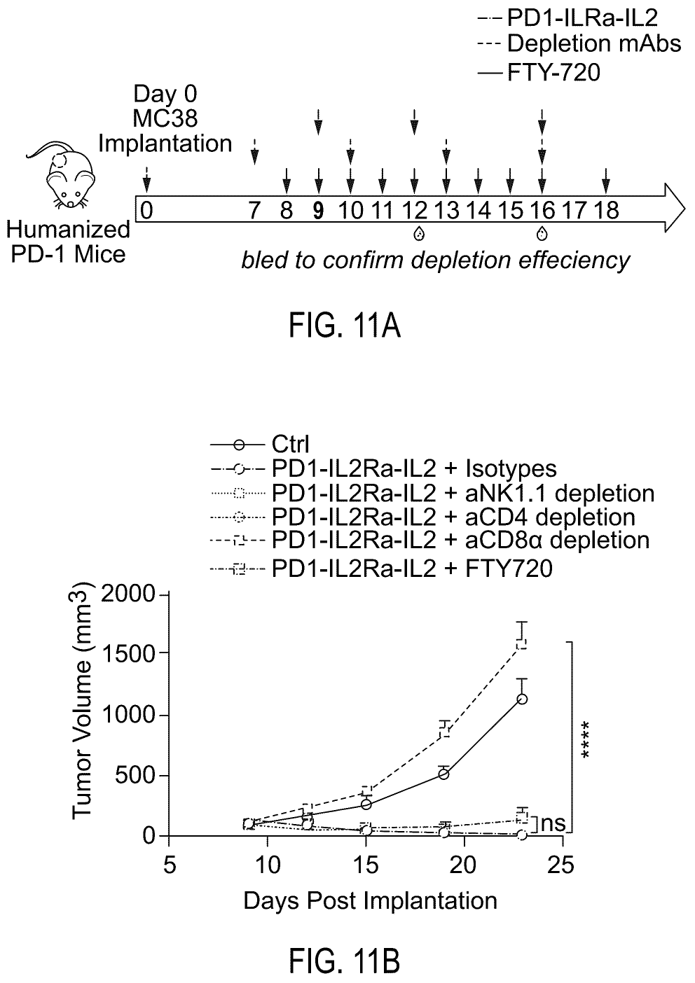

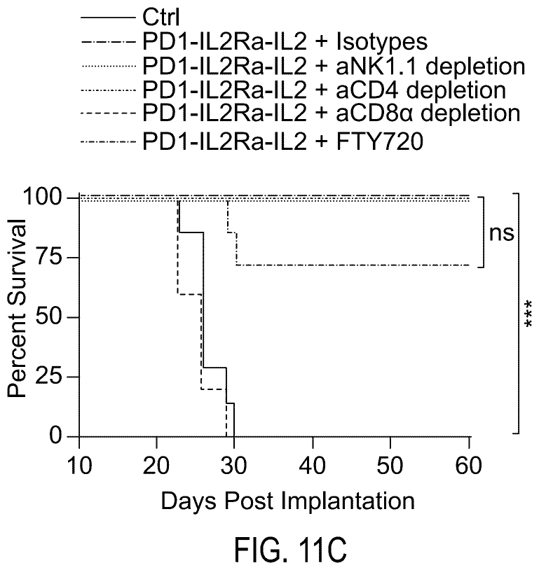

To determine the lymphocyte subsets required for the anti-tumor efficacy of PD1-IL2Ra-IL2, NK cells, CD8+ or CD4+ T cells were depleted prior to and during PD1-IL2Ra-IL2 treatment (FIG. 11A). Human PD-1 knock-in mice bearing established MC38 tumors were randomized (5-7 mice per group) and were treated with isotype control or PD1-IL2Ra-IL2 (0.5 mg/kg) in combination with the indicated depletion mAbs or FTY-720 via intraperitoneal injection. Schedule of injection for each reagent is indicated on the schematic (FIG. 11A). Tumor growth (mean±SEM) (FIG. 11B) and mice survival (FIG. 11C) were monitored over time. Statistical analyses for tumor growth were performed using two-way ANOVA with Bonferroni's multiple comparisons tests (*P≤0.05, **P≤0.01, ***P≤0.001, ****P≤0.0001). All statistical significance for mice survival were determined by Kaplan-Meier analyses with the log-rank tests (***P≤0.001, ****P≤0.0001).

Depletion of CD8+ T cells, but not CD4+ T cells or NK cells, completely abrogated the tumor growth inhibition and tumor-free survival induced by PD1-IL2Ra-IL2 (FIGS. 11A-11C). In addition, most of this anti-tumor effect was retained when T-cell egress from lymph nodes was blocked via treatment with S1PR inhibitor FTY720, suggesting the pre-existing intratumoral CD8+ T cells play a more critical role in mediating the anti-tumor immunity elicited by PD1-IL2Ra-IL2 over cells newly recruited to the tumor.

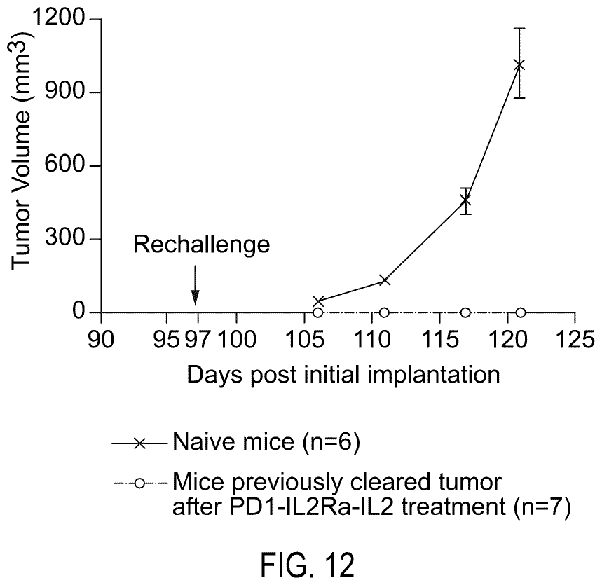

To further test whether PD1-IL2Ra-IL2 therapy generates anti-tumor memory, human PD1 knock-in mice that previously eradicated MC38 tumors after PD1-IL2Ra-IL2 treatment were rechallenged with MC38 tumors 97 days after initial tumor implantation (88 days after the last PD1-IL2Ra-IL2 dosing), and control naïve mice were challenged in parallel. PD-1 knock-in mice previously cleared of MC38 tumors after treatment with PD1-IL2Ra-IL2 were rechallenged with a secondary MC38 tumor implant through subcutaneous injection on the opposite flank. Tumor growth (mean±SEM) was monitored over time. Data are representative of three experiments. Statistical analyses for tumor growth were performed using two-way ANOVA with Bonferroni's multiple comparisons tests (*P≤0.05, **P≤0.01, ***P≤0.001, ****P≤0.0001). Whereas naïve mice developed progressive MC38 tumors, no tumor growth occurred in mice previously cured by PD1-IL2Ra-IL2 (FIG. 12). Collectively, these results demonstrate that PD1-IL2Ra-IL2 elicits robust anti-tumor immunity primarily by boosting intratumoral CD8+ T cell responses, which also results in the development of long-term memory against tumor cells.

PD1-IL2Ra-IL2 induces dynamically differentiating PD1+CD8+ effector T cells in the tumor: To gain deeper insight into the impact of PD1-IL2Ra-IL2 treatment on the immune landscape of the tumor microenvironment, single-cell RNA sequencing (scRNA-seq) was performed of CD45+ immune cells isolated from MC38 tumors after treatment with PD1-IL2Ra-IL2, NT-IL2Ra-IL2, anti-PD1 or isotype control, respectively. Globally, PD1-IL2Ra-IL2 led to a pronounced expansion of T cell populations and a reduction in myeloid populations relative to other treatment groups.

Further unsupervised clustering of all CD3e+ T cells identified four CD4+ T cell clusters (CD4_1 to CD4_4) and nine CD8+ T cell clusters (CD8_1 to CD8_9). Among the CD4+ T cell clusters, PD1-IL2Ra-IL2 treatment did not increase the size of FoxP3+ Treg populations (CD4_1, CD4_2 and CD4_3), while slightly expanded CD4_4 which expressed genes associated with a non-lymphoid tissue memory T cell identity (Ramp3, Tnfsf8, Tnfsf11) (Miragaia, 2019). The predominant impact of PD1-IL2Ra-IL2 treatment was on the CD8+ T cells, where it induced expansion of several PD1+CD8+ T cell clusters (CD8_4, CD8_5, CD8_6, CD8_8 and CD8_9) that were absent or minimally presented in other treatment groups. These clusters all displayed upregulated, despite at variable levels, expression of genes linked to effector functions (Gzma, Klrd1, Prf1, Cx3cr1, Ifng) and activation/exhaustion makers (Pdcd1, Lag3, II2Ra, Havcr2), representing effector CD8+ T cell populations transitioning between different activation and differentiation states. CD8_6 had the highest expression of multiple Gzm family genes and might stand for more differentiated cytotoxic effectors. Compared to CD8_6, CD8_4 had lower expression of Gzm genes, Prf1, and Havcr2, contained a small subset of cells expressing Tcf7, therefore might correspond to earlier effectors. CD8_5 had a similar level of effector gene expression to CD8_4 but also showed an interferon-stimulated gene signature (Isg15, Ifit1, Ifit3). CD8_8 and CD8_9 also expressed cell cycle genes (Mki67, Top2a, Stmn1), representing actively proliferating effector cells. Besides the predominant expansion effector CD8+ T cells, PD1-IL2Ra-IL2 treated group also showed a moderate enrichment of CD8_2, which co-expressed Pdcd1, Tcf7 and pro-survival gene Bcl2 and likely represented stem-like progenitor CD8+ T cells (Siddiqui, 2019; Miller, 2019). Consistently, pseudotime analysis of the CD8+ T cell clusters inferred a continuous differentiation trajectory starting from CD8_1, progressing to CD8_2 and 3, further to CD8_4 and 5, followed by CD8_6, and ending with CD8_7. Together, these results suggest PD1-IL2Ra-IL2 expands intratumoral PD1+ CD8+ T cells with robust effector profiles and diverse differentiation states, which is conducive to a robust and durable anti-tumor immunity.

To understand the clonal structure of different T cell clusters as well as the relationship between them, parallel single-cell TCRseq was performed with the same TIL samples analyzed by scRNA-seq. The effector CD8+ T cell clusters induced by PD1-IL2Ra-IL2 treatment showed the highest degree of clonal expansion, indicative of tumor-associated antigen recognition. In addition, considerable clonotype overlaps were observed among different expanded effector CD8+ T cell clusters, further corroborating the result from trajectory analysis and highlighting the ability of PD1-IL2Ra-IL2 to promote a dynamically differentiating tumor-reactive T cell compartment.

The ability of PD1-IL2Ra-IL2 to expand tumor antigen-specific T cells was confirmed using the TRAMP-C2 model, in which tumor rejection can be mediated by CD8+ T cells recognizing a tumor-specific antigen epitope Spas-1 (Fassò (2008)). C57BL/6 mice were implanted subcutaneously with TRAMP-C2 tumor cells. When tumors were established at 175 mm³, mice were grouped (4 mice per group) and treated with a single dose of indicated molecules via intraperitoneal injection. 6 days post dosing, mice with euthanized and tumors were excised and weighed. Tumors were dissociated into single-cell suspensions and analyzed by flow cytometry for tumor CD8⁺ T cell densities and SPAS-1-Dextramer⁺ CD8⁺ T cells as a percentage of total CD8⁺ T cells. SPAS-1-Dextramer⁺ CD8⁺ T cell density in the tumor was quantified. PD1⁺ CD8⁺ and TCF1⁺PD1⁺ CD8⁺ T cell densities in the tumor were quantified. Statistical analyses were performed using one-way ANOVA with Dunnett's multiple comparisons tests (**P≤0.01). Data are representative of two experiments.

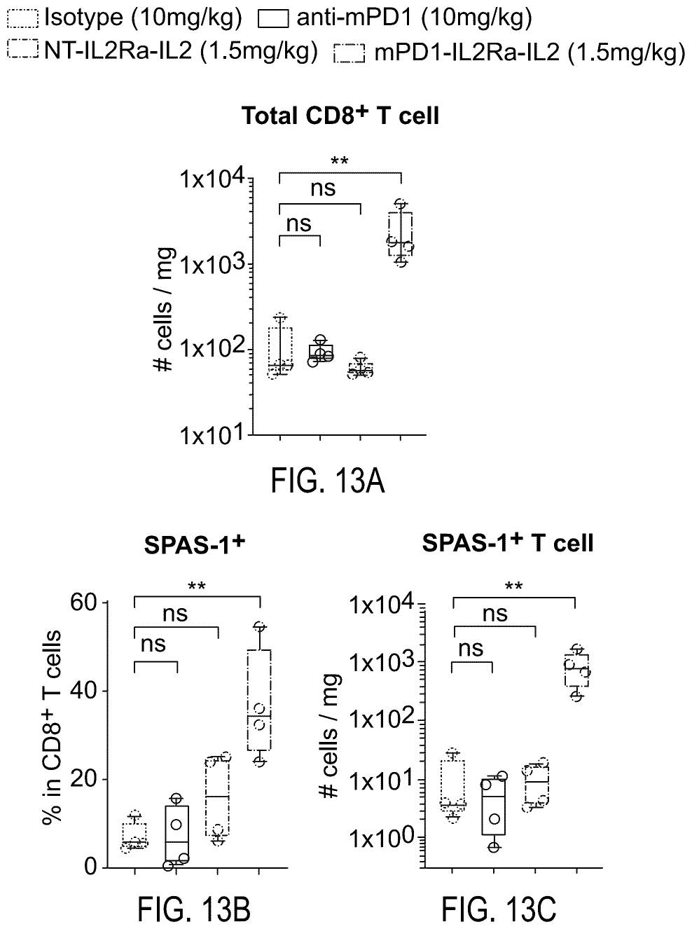

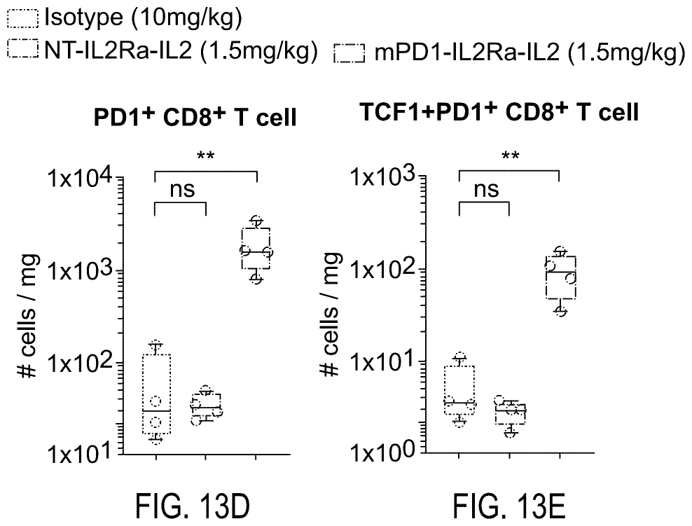

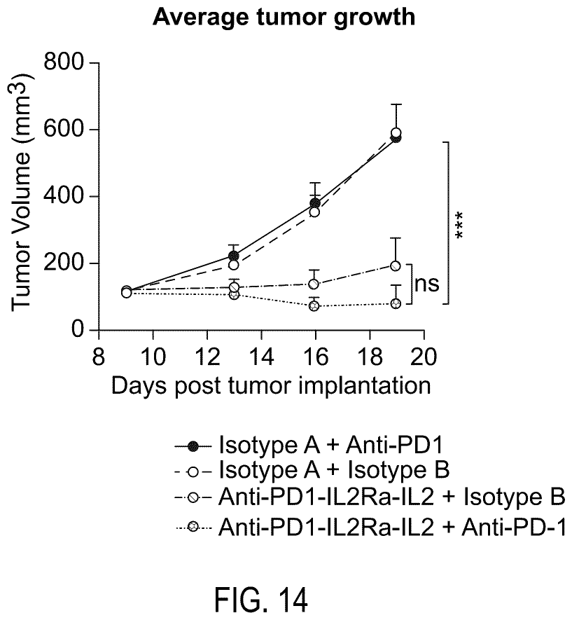

In accordance with the anti-tumor activity of PD1-IL2Ra-IL2 in the TRAMP-C2 model (FIG. 9C), flow cytometry analysis of TILs isolated from TRAMP-C2 tumors revealed a marked expansion of CD8+ T cells after treatment with PD1-IL2Ra-IL2 but not NT-IL2Ra-IL2 or a higher dose of anti-PD1 (FIG. 13A). Among the CD8+ T cells expanded, substantial increases in both the number and the frequency of Spas-1-specific CD8+ T cells were induced by PD1-IL2Ra-IL2, directly demonstrating its capacity to expand tumor-specific T cells (FIGS. 13B-13C). Consistent with PD1-IL2Ra-IL2 delivering IL-2 in a PD1-targeted manner, the vast majority of the expanded CD8+ T cells are PD1+, including a subset that is PD1+ TCF1+ and likely represents the stem-like progenitor pool of tumor-reactive T cells (FIGS. 13D-13E).

### Example 3: Improved Anti-Tumor Efficacy of Anti-PD1-IL2Ra-IL2 when Combined with Anti-PD1 in MC38/hPDL1 Model

This example relates to an in vivo study conducted to demonstrate the improved anti-tumor efficacy of the targeted immunocytokine, anti-PD1-IL2Ra-IL2 (or “PD1-IL2Ra-IL2”), when combined with the PD1-blocking anti-PD1 antibody, REGN2810 (cemiplimab), in treating established MC38 tumors, which are a murine model of colorectal cancer. Mice were humanized for PD1, i.e., mouse PD1 was knocked out and replaced with human PD1 (“human PD1 knock-in”).

On Day 0, human PD1 knock-in mice were implanted subcutaneously with MC38 tumor cells. On Day 9, when tumors reached an average tumor volume of approximately 120 mm³, mice were randomized (9-10 per group). Because both anti-PD1-IL2Ra-IL2 and cemiplimab target PD1, a staggered dosing regimen was used to minimize any potential interference between the two agents. Mice received a single dose of either anti-PD1-IL2Ra-IL2 or its matched isotype control (isotype A) on Day 9, followed by two injections of either cemiplimab or its corresponding isotype control (isotype B) on Days 14 and 19. Therefore, the group that received “anti-PD1-IL2Ra-IL2+isotype B” was considered as an anti-PD1-IL2Ra-IL2 monotherapy group, and the group that received the “isotype A+anti-PD1” would be equivalent to an anti-PD1 monotherapy group. Average tumor growth (mean±SEM) was monitored over time. Statistical analysis was performed using two-way ANOVA with Bonferroni's multiple comparisons tests (***P≤0.001). Statistical significance for mice survival was determined by Kaplan-Meier analyses with the log-rank tests (**P≤0.01, ****P≤0.0001). Data are representative of three independent experiments.

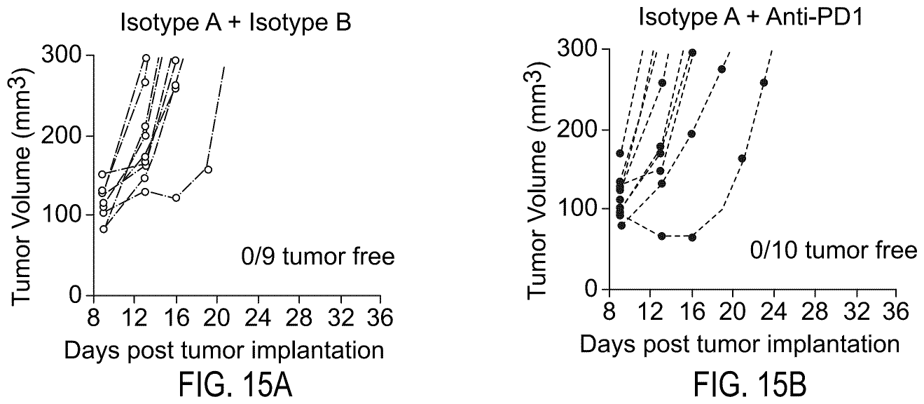

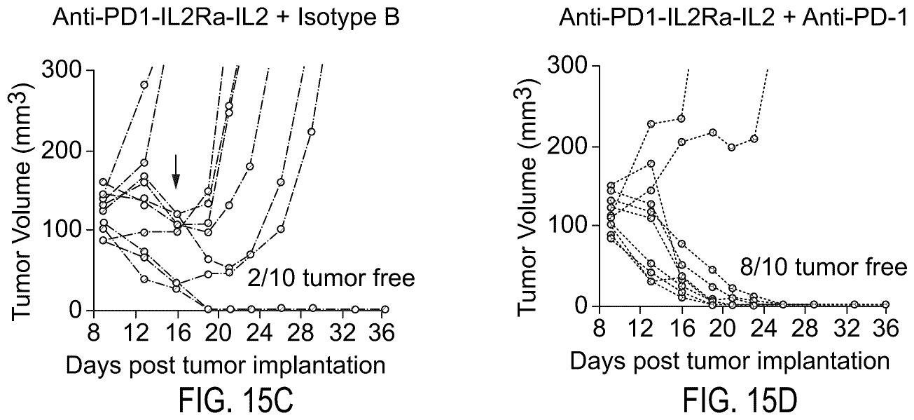

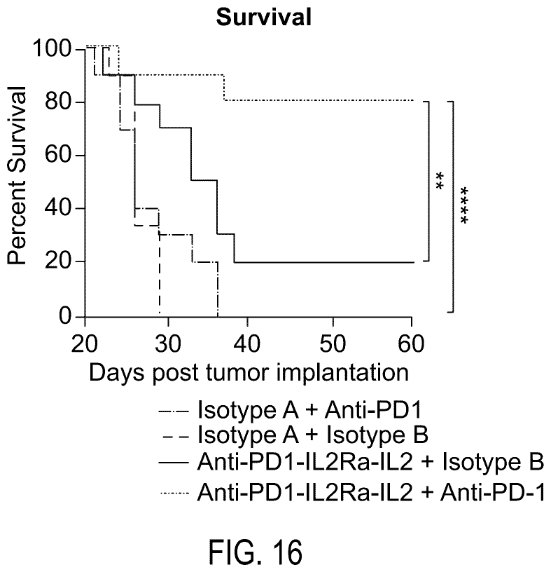

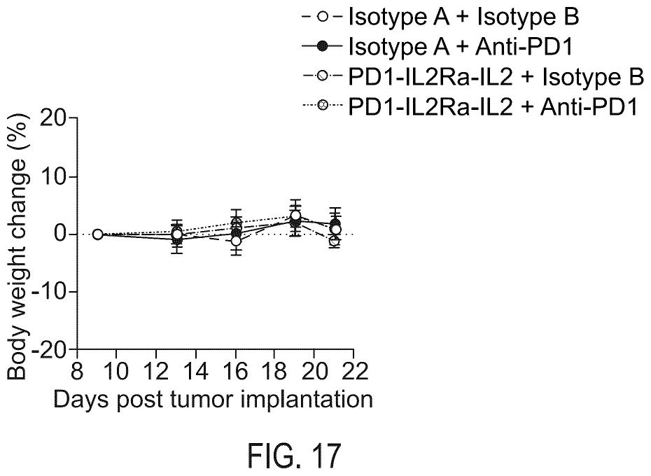

Results: A single dose of anti-PD1-IL2Ra-IL2 at 0.2 mg/kg initially triggered efficient tumor growth control, with 8 out of 10 mice in the anti-PD1-IL2Ra-IL2 monotherapy group undergoing tumor regression within the first week of treatment (FIG. 15C, arrow). However, most of these initial responders eventually relapsed (6 out of 8), leaving only 2 mice with complete tumor regression and long-term survival in the anti-PD1-IL2Ra-IL2 monotherapy group (FIG. 15C and FIG. 16). In contrast, mice that received anti-PD1-IL2Ra-IL2+anti-PD1 combination therapy showed significantly more durable responses, with a higher frequency (8 out of 10) of mice achieving complete tumor regression and long-term survival (FIG. 15D, FIG. 16). No tumor clearance or survival benefit was observed in groups that received the isotype control, or anti-PD1 monotherapy (FIGS. 15A-15B and FIG. 16). The combination of PD1-IL2Ra-IL2 with anti-PD1 did not result in any body weight loss in mice (FIG. 17).

Conclusions: Substantially improved anti-tumor efficacy was achieved by the targeted immunocytokine, anti-PD1-IL2Ra-IL2, when combined with the PD1-blocking anti-PD1 antibody, cemiplimab. The combination therapy was far superior to either monotherapy in reducing tumor growth, inducing complete tumor regression, increasing durability of tumor control, and improving long-term survival.

### Example 4: Materials and Methods

The following disclosure relates to the materials and methods used in the experiments described in Examples 2 and 3.

Protein production: All antibody or Fc/IL-2 fusion proteins and CD3 BsAbs were produced at Regeneron Pharmaceuticals. The proteins were expressed in either transient transfected Expi293F™ cells (ThermoFisher Scientific, A14527) or stably transfected Chinese hamster ovary cells, and subsequently purified using protein A-affinity capture followed by size exclusion chromatography or ion exchange and hydrophobic interaction chromatographies. Generation of MUC16×CD3 BsAb (REGN4018) and anti-PD-1 mAb (Cemiplimab) have been described previously (Burova, 2017; Young, 2022). The parental anti-human PD-1 antibody for anti-hPD1-IL2Ra-IL2 (REGN10597) was generated via immunization of VelocImmune® mice (Macdonald et al., Proceedings of the National Academy of Sciences, 111:5147-5152 (2014); Murphy et al., Proceedings of the National Academy of Sciences, 111:5153-5158 (2014)) with recombinant human PD-1-mFc protein.

Cell lines: Human T/NK cell leukemia YT cell line (DSMZ ACC-434) was used to create a series of YT/STAT5-Luciferase reporter cell lines with different expression levels of IL-2Ra and PD-1. For generating a parental YT/STAT5-Luc cell line, YT cells was electroporated with a STAT5 response element driven luciferase reporter construct (Promega, #E4651), single cell cloned and maintained in Iscove's Medium (Irvine Scientific) supplemented with 20% FBS, 1× L-glutamine, Penicilin/Streptomycin (Gibco™, 10378016) and 200 μg/mL Hygromycin-B. To further generate YT/STAT5-Luc/IL2RaKO and/or PD1KO cell lines, IL-2Ra (CD25) and/or PD-1 were knocked out using TrueCut Cas9 Protein v2 (ThermoFisher Scientific, A36498) with a guide targeting either IL-2Ra (ThermoFisher Scientific, CRISPR1029141_SGM, target sequence AATGTGGCGTGTGGGATCTC) or PD-1 (ThermoFisher Scientific, CRISPR816555_SGM, target sequence ATGTGGAAGTCACGCCCGTT). IL-2Ra and/or PD-1 negative cells were isolated by FACS. To generate IL-2RαOE and/or PD1OE derivatives of the above-mentioned cell lines, human IL-2Rα and/or PD-1 were stably expressed by lentiviral transduction with a vector encoding IL-2Rα (M1-1272 of accession number NP_000408.1) and/or PD-1 (M1-L288 of accession number NP_005009.2). Cells were selected in media supplemented with 15 μg/ml blasticidin (for IL-2Ra selection) and/or 1 mg/mL G418 (for PD-1 selection). Human IL-2Ra and/or PD-1 positive cells were validated by FACS.

MC38 (from National Cancer Institute Lab of Tumor Immunology and Biology), B16F10 (ATCC, CRL-6475), MCA205 (Sigma-Aldrich, SCC173) and TRAMP-C2 (ATCC, CRL-2731) tumor cells were cultured in DME (Irvine Scientific, 9031) media supplemented with 10% FBS (Seradigm, 1500-500) and 1× L-glutamine/Penicillin/Streptomycin (Gibco™, 10378016). Colon26 (ATCC, CRL2638) cells were cultured in RPMI1640 (Irvine Scientific, 9160) medium supplemented with 10% FBS and 1× L-glutamine, Penicillin/Streptomycin. The ID8-VEGF cell line (Ludwig Institute for Cancer Research) was maintained in DME media supplemented with 10% FBS and 1× L-glutamine, Penicillin/Streptomycin. The ID8-VEGF/huMUC16-delta cell (Crawford, 2019) was generated by stably expressing the membrane-proximal region of human MUC16 on the parental ID8-VEGF cell.

Cell surface receptor binding assay: Binding of PD1-IL2Ra-IL2 and control molecules to cell surface IL-2 receptor complexes were evaluated by flow cytometry. Briefly, cells were incubated with a serial dilution of each indicated molecule at 4° C. for 30 min, followed by two washes with FACS buffer (PBS+2% FBS). Bound molecules were detected with AlexaFluor 647-conjugated anti-human IgG H/L secondary F(ab′)2 antibodies (Jackson Immunoresearch, 109-606-088) in the presence of fixable violet Live/Dead dye (Invitrogen, #L34960). Cells were washed twice, fixed, and acquired on an iQue Screener Plus flow cytometer (IntelliCyt) and analyzed using Forecyt software. Geometric mean fluorescence intensities were plotted, with a no titrated molecule condition (secondary antibody only) represented as the lowest point on the titration curve.

STAT5-luciferase reporter bioassay: Engineered YT/STAT5-Luciferase reporter cell lines with different expression levels of IL-2Ra and PD-1 were incubated with serial dilutions of IL-2 (Proleukin, Clinigen #NDC7631D-022-01), PD1-IL2Ra-IL2, parental PD-1 mAb or NT-IL2RA-IL2 control at 37° C. for 4.5 hours. Following incubation, One-Glo luciferase assay reagent (Promega, #E6130) was added and luminescence signal was measured on an Envision plate reader. Relative Light Units (RLU) were plotted, with a no titrated molecule condition represented as the lowest point on the titration curve. All samples were tested in triplicates.

STAT5 phosphorylation assay on human PBMCs: Human peripheral blood mononuclear cells (PBMCs) were obtained from BiolVT, LLC. Cryopreserved human PBMCs were thawed and rested overnight in media without IL-2. The next day, cells were stained with fixable Near-IR viability dye (Invitrogen) for 15 min at room temperature. Cells were washed and treated with serial dilutions of indicated molecules for 20 min, then fixed with BD Cytofix™ fixation buffer at 37° C. for 12 min. Cells were then permeabilized with pre-chilled BD Phosflow™ Perm Buffer III for 10 min on ice. Cells were washed twice with FACS buffer (PBS+2% FBS), followed by staining with Alexa Fluor® 647-conjugated anti-Stat5 pY694 antibody (BD Biosciences-562076) at room temperature for 60 min in the dark. To identity different lymphocyte populations in PBMCs, the following panel of antibodies were also included in the staining cocktail: CD8 (RPA-T8), CD4 (L200), CD25 (MA251), CD56 (HCD56), CD3 (SP34-2), Foxp3 (259D/C7), PD-1 (EH12.1) and NKp46 (9-E2). Cells were washed twice with FACS buffer before data acquisition on a BD LSRFortessa™ X-20 flow cytometer. The raw data were analyzed with FlowJo.

Mixed lymphocyte reaction (MLR) assay: MLR assay was performed to assess primary human T cell activation in the presence of allogeneic PBMC. PBMC were isolated from blood using the EasySep™ Direct Human PBMC Isolation Kit (STEMCELL Technologies) according to the manufacturer's protocol. T cells were isolated from PBMC using the EasySep™ T-Cell Isolation Kit (STEMCELL Technologies) according to the manufacturer's protocol. Allogeneic PBMC that were used to stimulate allogeneic T cells, were incubated for 1 hour at 37° C. and 5% CO₂ in assay media (X-Vivo 15, 10% FBS, HEPES, sodium pyruvate, nonessential amino acids, 0.01 mM beta-mercaptoethanol) with 50 μg/mL mitomycin C to arrest cell growth. Growth arrested allogeneic PBMC were washed with assay media and incubated with T cells for 6 days at 37° C. and 5% CO₂. The pre-stimulated T cells were isolated from the co-culture containing the growth-arrested PBMC using the Easy Sep T Cell Isolation Kit according to the manufacturer's protocol, resuspended in assay media, and rested for 24 hours at 37° C. and 5% CO2 prior to the assay.

As pre-stimulated T cells were resting, new vials of allogenic PBMC were thawed and resuspended in assay media containing 50 ng/ml IFNγ and incubated overnight at 37° C. and 5% CO₂. The following day, the IFNγ-treated PBMC were treated with 50 μg/ml mitomycin C in assay media, for 1 hour at 37° C. and 5% CO₂, and then washed with Dulbecco's PBS containing 2% FBS prior to the assay.

Pre-stimulated T cells and growth-arrested, IFNγ-treated allogeneic PBMC were added to plates at a ratio of 1:3 T cells: PBMC. Cells were incubated with serial dilutions of Proleukin, PD1-IL2Ra-IL2, parental PD-1 mAb, NT-IL2RA-IL2 or isotype control 72 hours. Supernatant from the assay plates was removed to perform the AlphaLISA human TNFα and IFNγ assays according to the manufacturer's protocol. Plates were read on an Envision multimode plate reader. Measured TNFα and IFNγ concentrations were plotted, with a no titrated molecule condition represented as the lowest point on the titration curve. All samples were tested in triplicates.

Animal studies: All animals were maintained under pathogen-free conditions. C57BL/6 and BALB/c mice were obtained from the Jackson Laboratory. Human PD-1 knock-in mice and human CD3/MUC16 knock-in mice were generated in-house and described previously (Burova, 2017; Crawford, 2019). All mice were 8-16 weeks old when used in the studies.

For tumor studies, unless otherwise indicated, different mouse strains were implanted with tumor cells resuspended in HBSS (Gibco™ 14025076) by subcutaneous injection on the right flank. The following tumor cell numbers were used: MC38 (3×10⁵ cells), B16F10 (3×10⁵ cells), MCA205 (1×10⁶ cells), TRAMP-C2 (1×10⁶ cells), Colon26 (1×10⁶ cells). When tumors were established, mice were randomized based on tumor size and treated with indicated molecules and doses on the indicated days via intraperitoneal injection. For the ID8-VEGF/huMUC16-delta studies, human CD3/MUC16 knock-in mice were subcutaneously implanted with 1×10⁷ tumor cells, resuspended in RMPI1640 medium, and treatments were administered via intravenous injection. Tumor growth was monitored over time using caliper measurements and calculated as (length×width²)/2. Mice were euthanized when tumors reached >2000 mm³ or became severely ulcerated. For tumor rechallenge experiments, 3×10⁵ MC38 cells or 1×10⁷ ID8-VEGF cells were implanted on the left flanks of corresponding tumor-free mice more than 75 days after they received and rejected their primary tumor challenges.

For immune cell depletion studies, mAbs used were as follows: mouse IgG2a isotype control (300 ug) clone C1.18.4 (BioXCell-BE0085), rat IgG2b isotype control (500 ug), clone LFT-2 (BioXCell-BE0090), anti-NK1.1 (300 ug) clone PK136 (BioXCell-BE0036), anti-CD4 (500 ug) clone GK1.5 (BioXCell BE0003-1), anti-CD8 (300 ug) clone 2.43 (BioXCell-BE0061). Depletion mAbs were administered by intraperitoneal injection 2 days before initiation of PD1-IL2Ra-IL2 dosing. Depletion mAbs were administered twice a week for a total of 4 doses. PD1-IL2Ra-IL2 (0.5 mg/kg) was administered every 3-4 days for a total of 3 doses. FTY-720 (Sigma-SML0700-25 MG) was diluted in saline and administered by intraperitoneal injection 1 day before initiation of PD1-IL2Ra-IL2 dosing. FTY-720 (60 ug) was administered based on the dosing schema in FIG. 11A. Blood was collected twice over two weeks to verify expected depletion of peripheral lymphocyte population.

CAR-T cell generation and in vivo efficacy: To generate murine anti-huMUC16 CAR T cells, CD3+ T cells were isolated from the spleens of huCD3/huMUC16 knock-in mice using an untouched TM mouse T-cell isolation kit (Invitrogen #11413D) before activation with CD3/CD28 Dynabeads (Invitrogen #11161D) and recombinant human IL-2 (20 U/ml; Peprotech #200-02). After 16 hours, the T-cells were transduced via spin-infection on plates coated with Retronectin (Takara #T100B) with retrovirus encoding an anti-huMUC16 CAR containing murine CD3z and human 4-1BB intracellular signaling domains. CAR T cells that bind an irrelevant antigen were generated in parallel and used as controls. To assess PD-1 expression by the CAR T cells in the presence of target cells expressing cognate antigen, anti-huMUC16 CAR T cells or control CAR T cells were mixed with either ID8-VEGF or ID8-VEGF/huMUC16-delta tumor cells at a 1:1 ratio for 16 hours, followed by flow cytometry analysis of PD-1 expression on gated CAR+ cells.

To study the combination of PD1-IL2Ra-IL2 and CAR T cell therapy, huCD3/huMUC16 knock-in mice were lymphodepleted via a sublethal dose of total body irradiation (400 cGy) one day before subcutaneous implantation with 1×10⁷ ID8-VEGF/huMUC16-delta tumor cells in the right flank. One day after tumor implantation, mice were injected intravenously with 4×10⁶ anti-huMUC16 CAR T or control CAR T cells. The same day mice were intraperitoneally dosed with isotype (10 mg/kg), anti-mPD1 (10 mg/kg), NT-IL2Ra-IL2 (1 mg/kg) or mPD1-IL2Ra-IL2 (10 mg/kg). Two days post CAR T cell injection, the mice received one additional dose of the same protein molecules. Tumor volume was measured twice weekly by using calipers and calculated as (length×width²)/2.

Immunophenotyping by flow cytometry: For flow cytometry analysis of lymphocyte populations, blood, spleens and tumors from treated mice were collected on indicated days. Blood samples were subjected to red blood cell lysis using ACK buffer (Lonza Bioscience) for 5 min on ice, quenched with complete RPMI, and washed twice prior to staining. Spleens were mashed through 100 μm Corning® cell strainers followed by red blood cell lysis. Tumors were excised and weighed before mincing. Fragments were digested into single-cell suspensions using the Miltenyi tumor isolation kit (130-096-730).

Cells were first stained with fixable Near-IR LIVE/DEAD dye (Invitrogen, #L34976) following the manufacturer protocol, followed by cocktails of fluorophore conjugated antibodies for cell-surface and intracellular markers. Staining antibodies were diluted in BD Horizon Brilliant Buffer (BD/563794) and surface staining was performed for 30 minutes on ice in the presence of 2% normal rat serum (ImmunoReagents—SP-006-VX2), 2% normal mouse serum (Jackson Immunoresearch—015-000-120), and 1:100 CD16/CD32 block (BD—553141). Antibody clones used for staining are: CD45 (30-F11), CD90.2 (53-2.1), CD8a (53-6.7 or CT-CD8a), CD4 (GK1.5 or RM4-4), CD19 (1D3), NK1.1 (PK136), NKp46 (29A1.4), Ki-67 (16A8), mPD-1 (29F.1A12), hPD-1 (EH12.1), CD44 (IM7), CD62L (MEL-14), Foxp3 (MF23), CD25 (PC61), TCF1 (C63D9), CD11b (M1/70), Ly6G (1A8), Ly6C (HK1.4). For dextramer staining, SPAS-1 Dextramer (Immudex—JA3973-PE) was first incubated with cells for 10 mins at room temperature before incubation with surface stain antibodies for 30 mins on ice. Intracellular staining was carried out using the eBioscience™ FoxP3 staining buffer set (Invitrogen 00-5523-00) according to the manufacturer's protocol. Stained samples were acquired on either BD LSR Fortessa X-20 or Cytek® Aurora flow cytometers and data were analyzed using FlowJo software.

Single cell RNA-seq and TCRseq: To prepare TIL samples for single cell RNA-seq and TCRseq, human PD-1 knock-in mice were subcutaneously implanted with MC38 tumor cells on both left and right flanks. When tumors were established, mice were randomized and treated on days 10 and 13 post tumor engraftment with the indicated molecules through intraperitoneal injection. On day 16 post tumor engraftment, mice were euthanized, and tumors were dissected and dissociated into single cell suspensions using a tumor isolation kit (Miltenyi 130-096-730). Right and left flank tumors from the same mouse were pooled. Single-cell suspensions were enriched for CD45+ tumor-infiltrating lymphocytes using positive selection using magnetic beads (Miltenyi 130-110-618) to achieve >95% CD45+ purity in live cells as assessed by flow cytometry (Miltenyi 130-110-797).

Surface plasmon resonance (SPR) assay for binding to IL-2 receptor proteins: SPR assays were performed on a Biacore S200 instrument (Cytiva™). To prepare a capture sensor surface, an anti-hFab capturing antibody (Cytiva™, Cat. #28958325) was immobilized on the surface of a CM5 sensor chip (Cytiva™, Cat. #BR-1005-30) using standard amine-coupling chemistry. The coupling procedure was performed using filtered HBSEP (10 mM HEPES, 150 mM NaCl, 3 mM ethylenediaminetetraacetic acid [EDTA], 0.05% (v/v) polysorbate-20, pH 7.4) as a running buffer. The sensor surfaces were activated by injecting a 1:1 (v/v) mixture of 0.4M 1-ethyl-3-(3-dimethylaminopropyl) carbodiimide hydrochloride and 0.1M N-hydroxysuccinimide over the chip surface at a flow rate of 30 μL/min for 7 minutes. Anti-hFab (20 μg/mL in 10 mM sodium acetate, pH 5.0) was injected over the activated chip surfaces at a flow rate of 30 μL/min for 7 minutes and a surface density of approximately 10,000 RU was reached. The remaining active groups on the CM5 sensor chip surface were blocked by injecting 1M ethanolamine at a flow rate of 30 μL/min for 7 minutes. The sensor chip surfaces were then washed with HBS-EP prior to performing the binding experiments.

The binding of recombinant hIL2RA (R&D Systems, Cat. #10305-RL-050), hIL2RB and hIL2RG proteins to PD1-ILR2-IL2 was measured at pH 7.4 and 25° C. using HBS-EP as running buffer. PD1-IL2Ra-IL2 or control reagents (parental PD-1 mAb, PD1-IL2) were captured by surface-coupled anti-hFab until a signal of approximately 400 to 600 RU was reached. A concentration of 1 uM of IL-2R proteins was selected as it was near equilibrium binding affinities and remained technically feasible. An “A-B-A” injection format was followed whereby recombinant IL2R proteins “A” were individually injected over the captured PD1-ILRa-IL2 or control reagent surfaces at a flow rate of 15 μL/min for 60 seconds, immediately followed by sample solution “B” (i.e., a second injection containing a mixture of IL-2 receptor components) for 60 seconds, then the flanking solution “A” for 60 seconds, followed by a 2-minute dissociation phase. The resultant binding signal changes were recorded, and the capture levels and binding responses for each of the injection steps was evaluated using Scrubber software.

Fab-arm exchange and mass spectrometry: aPD1 mAb, aPD1-IL2, and aPD1-IL2Ra-IL2 molecules were treated with PNGase F (New England Biolabs Inc), used at 1 IUB milliunit per 10 μg of protein, at 45° C. in 50 mM Tris-HCl (pH 7.0) for 1 hour to remove the N-glycan chains from each heavy chain CH2 domain. To set up Fab-arm exchange experiments, an aliquot of the deglycosylated aPD1 mAb was mixed with either deglycosylated PD1-IL2 or PD1-IL2Ra-IL2 molecule, and each corresponding mixture was subjected to limited reduction by 2 mM DTT (Pierce™, No-Weigh™ Format) in 50 mM Tris-HCl, pH 7.5 buffer (Thermo Fisher Scientific) at 37° C. for 30 min to only reduce inter-chain disulfide bonds.

The products of Fab-arm exchange were monitored by desalting size exclusion chromatography coupled to mass spectrometry (desalting SEC-MS) analysis. Desalting SEC was performed on an UltiMate 3000 UHPLC System (Thermo Fisher Scientific, Bremen, Germany) equipped with an Acquity BEH200 SEC guard column (4.6×30 mm, 1.7 μm, 200 Å; Waters, Milford, MA) with the column compartment set to 30° C. An isocratic flow of 150 mM ammonium acetate at 0.4 mL/mL was applied to perform online desalting prior to native MS analysis. To enable online native MS analysis, the analytical flow was split into a microflow (<10 μL/min) for nano-electrospray ionization (NSI)-MS detection and a remaining high flow for UV detection. A Thermo Q Exactive UHMR (Thermo Fisher Scientific, Bremen, Germany) equipped with a Microflow-Nanospray Electrospray Ionization (MnESI) Source and a Microfabricated Monolithic Multi-nozzle (M3) emitter (Newomics, Berkley, CA) was used for native MS analysis (Yan, 2020). Intact mass spectra from desalting SEC-MS analysis were deconvoluted using Intact Mass™ software from Protein Metrics.

### Example 5: Distinct Patterns of T Cell Expansion Between the Tumor and the Spleen in MC38.Ova Tumor-Bearing Mice Administered mPD1-IL2Ra-IL2

This example relates to Example 2, section “PD1-IL2Ra-IL2 exerts potent antitumor activity in syngeneic tumor models,” and shows that mice bearing MC38.Ova tumors display distinct patterns of T cell expansion between the tumor and the spleen in response to mPD1-IL2Ra-IL2 administration.

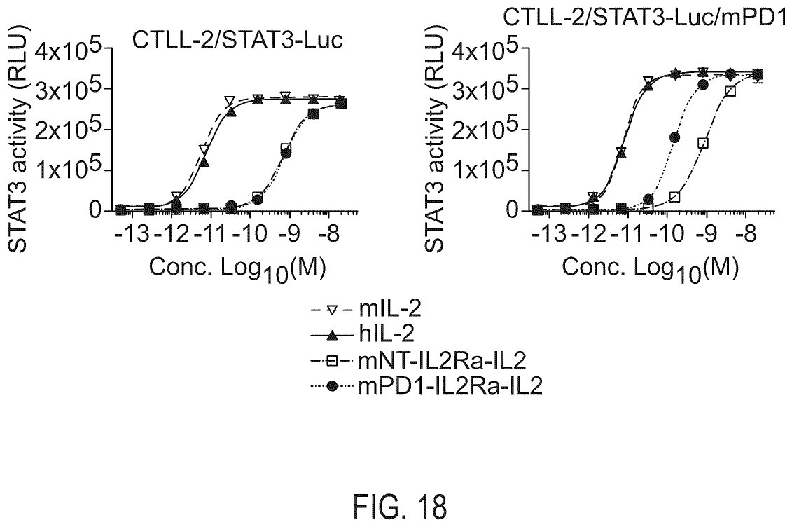

Characterization of the binding and activity of mouse surrogate mPD1-IL2Ra-IL2 molecule: As human IL-2 is highly cross-reactive on mouse cells (Mosmann et al., The Journal of Immunology 138, 1813-1816 (1987), the mouse surrogate molecule contains the same IL2Ra-IL2 moiety as the human construct but was instead fused to an anti-mouse PD1 antibody on mouse IgG1 isotype. Parental or mouse PD-1-overexpressed CTLL-2/STAT3-Luciferase reporter cells were incubated with a titration of each indicated molecule, STAT3 luciferase activity was assessed after 4.5 hr incubation. Compared to its isotype matched non-targeting mNT-IL2Ra-IL2 control (IL2Ra-IL2 fusion to an irrelevant anti-drug antibody), the mPD1-IL2Ra-IL2 surrogate molecule displayed binding to activated murine T cells and demonstrate PD-1-targeting enhanced activation of STAT3 signaling on CTLL-2 murine T cell lines (FIG. 18).

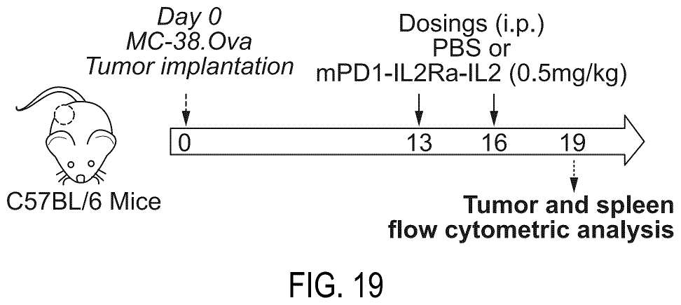

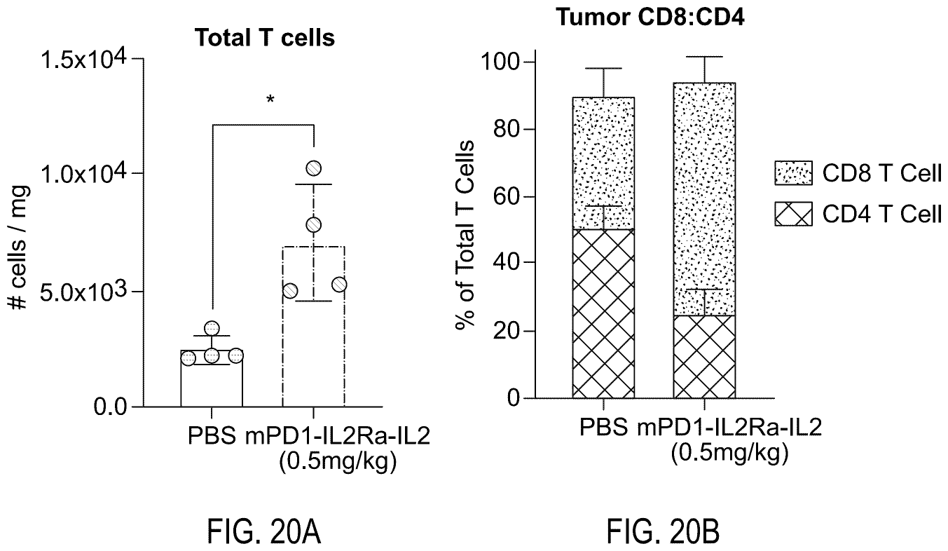

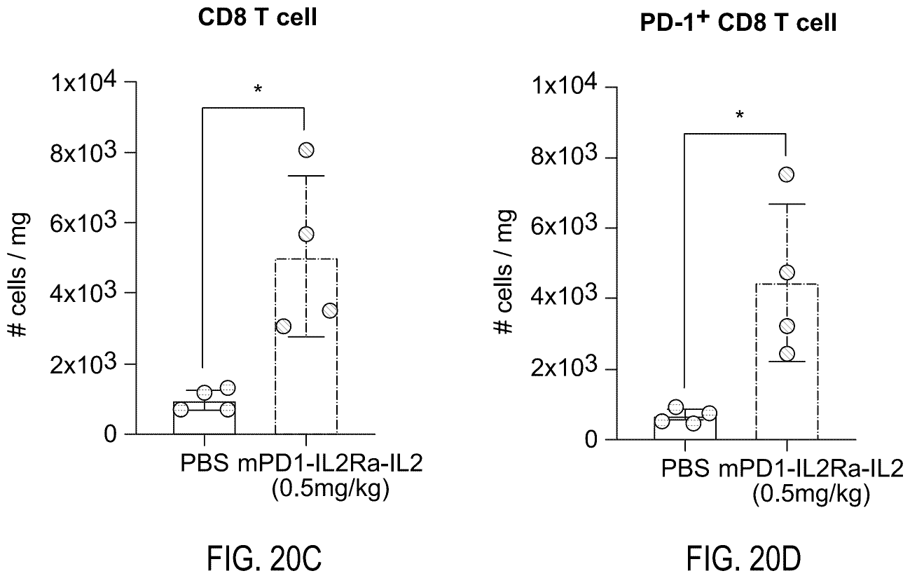

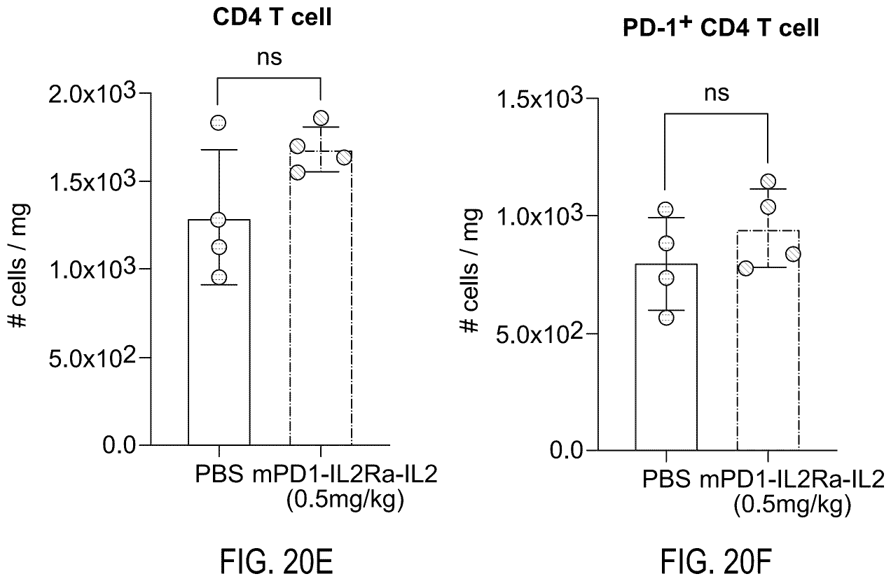

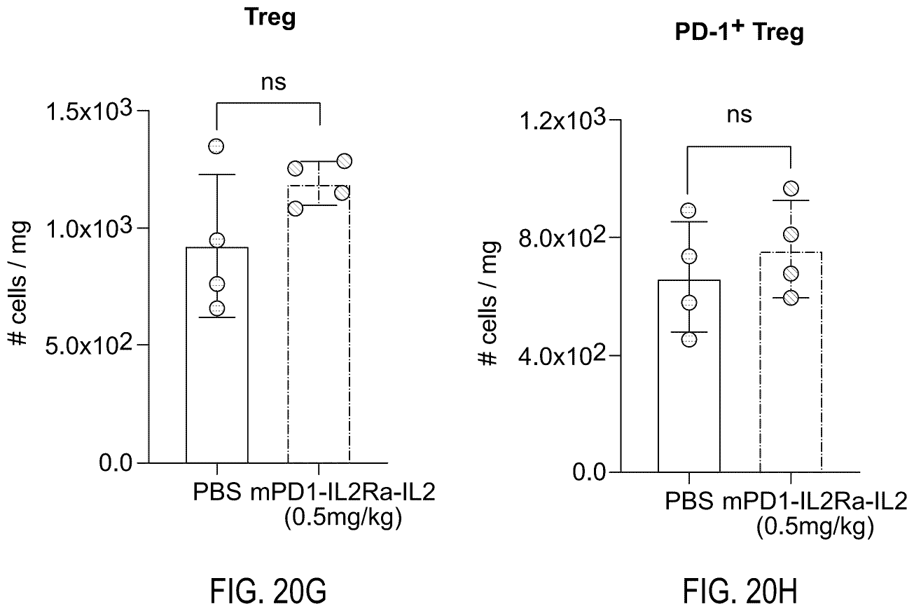

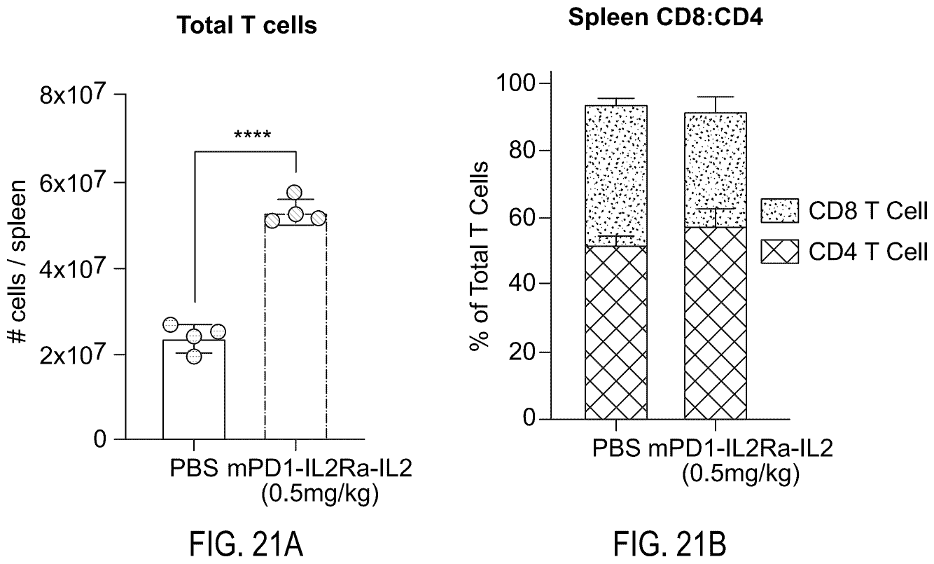

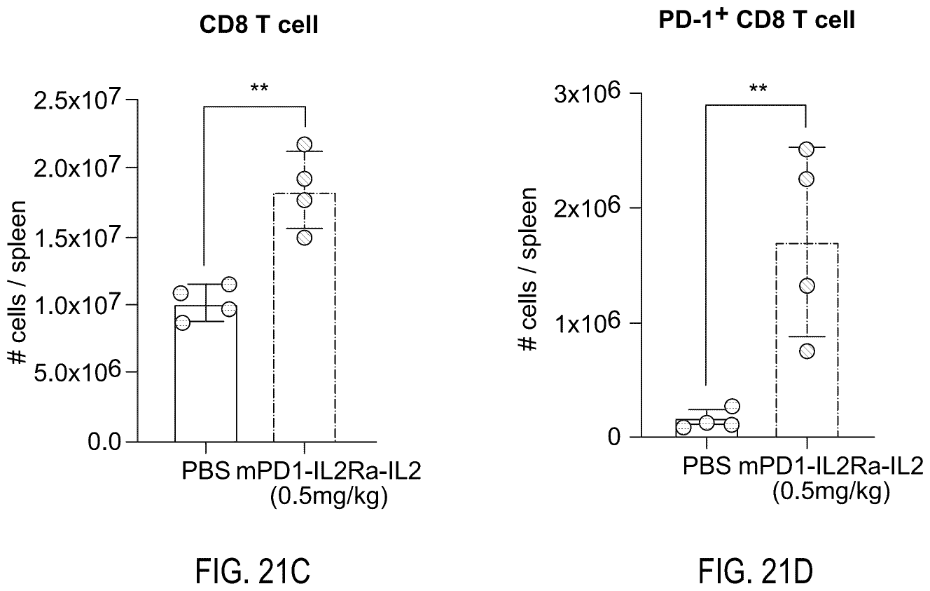

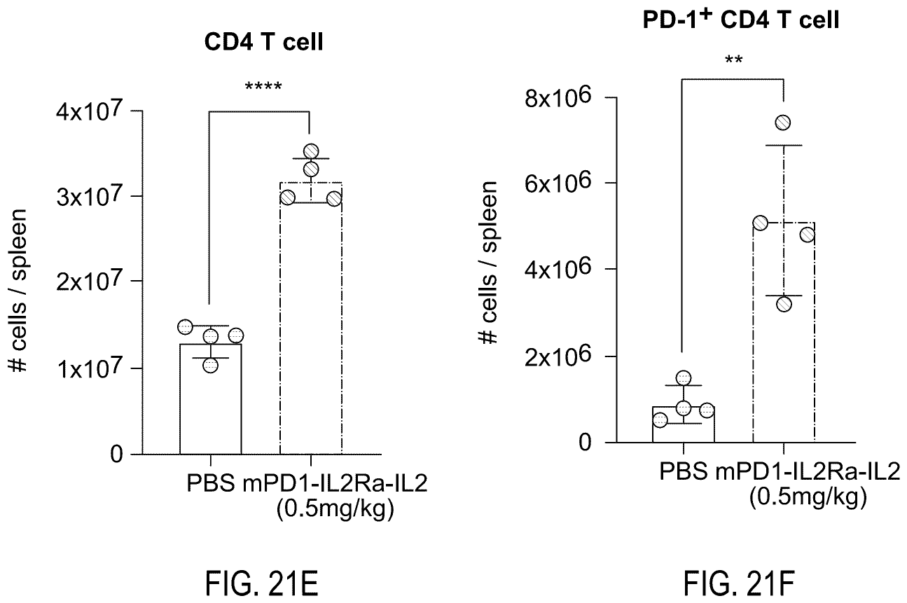

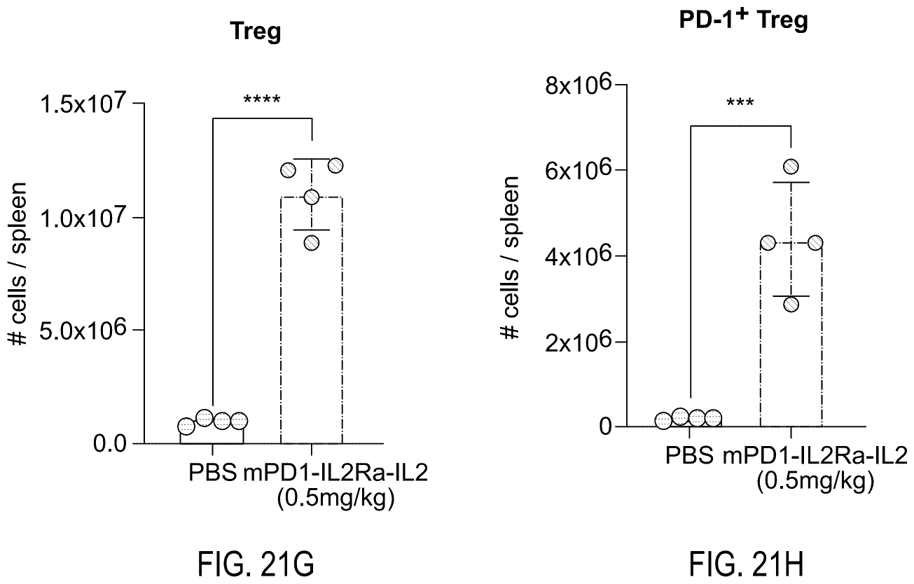

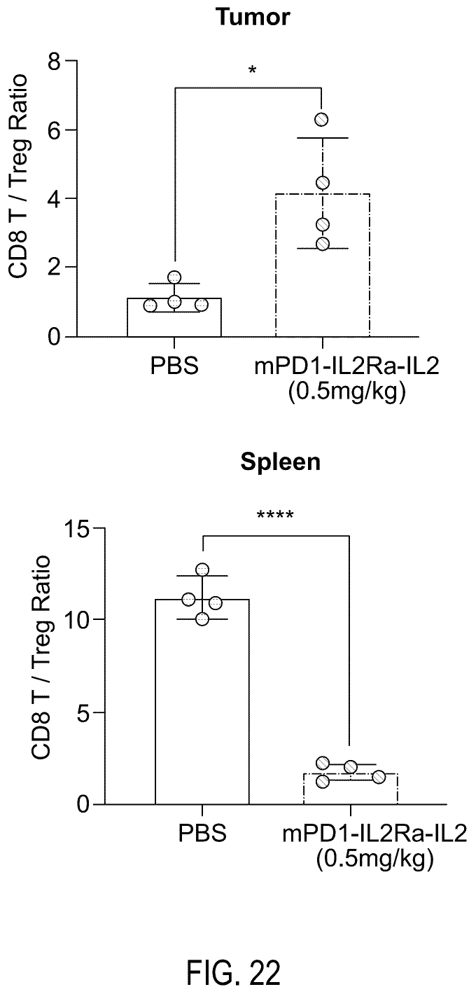

Flow cytometric analysis of MC38.Ova tumor-bearing mice following mPD1-IL2Ra-IL2 treatment: MC-38.Ova tumor cells (5×10⁵ cells) were engrafted subcutaneously into C57BL/6 mice and established before treatment with either PBS or mPD1-IL2Ra-IL2 on indicated days (FIG. 19). Tumors and spleens were harvested and dissociated for flow cytometric analysis (n=4 per group). Flow cytometric analysis of MC38.Ova tumor-bearing mice following mPD1-IL2Ra-IL2 treatment revealed distinct patterns of T cell expansion between the tumor and the spleen. In line with its potent anti-tumor activity, mPD1-IL2Ra-IL2 promoted the expansion of PD1+ CD8+ T cells but had minimal impact on Tregs in the tumor (FIGS. 20A-20H, 22). This contrasts with the immune cell profile observed in the spleen, where mPD1-IL2Ra-IL2 expanded both Tregs and PD1+ CD8+ T cells compared to the PBS control (FIGS. 21A-21H, 22).

### REFERENCES

Bagchi et al., Annual Review of Pathology: Mechanisms of Disease 16, 223-249 (2021)
June et al., Science 359, 1361-1365 (2018)
van de Donk et al., The Lancet 402, 142-158 (2023)
Waldman et al., Nature Reviews Immunology 20, 651-668 (2020)
Fridman et al., Nature Reviews Cancer 12, 298-306 (2012)
Waldmann, Cold Spring Harbor Perspectives in Biology 10 (2018)
Spolski et al., Nature Reviews Immunology 18, 648-659 (2018)
Rosenberg, The Journal of Immunology 192, 5451-5458 (2014)
Dutcher et al., Journal for ImmunoTherapy of Cancer 2, 26 (2014)
Vial, et al., Drug Safety 7, 417-433 (1992)
Charych et al., Clinical Cancer Research 22, 680-690 (2016)
Klein et al., Oncolmmunology 6, e1277306 (2017)
Lopes et al., Journal for ImmunoTherapy of Cancer 8, e000673 (2020)
Ptacin et al., Nature Communications 12, 4785 (2021)
Bentebibel et al., Cancer Discovery 9, 711-721 (2019)
Janku et al., Cancer Research 81, LB041-LB041 (2021)
Tzeng, et al., Proceedings of the National Academy of Sciences 112, 3320-3325 (2015)
van Brummelen et al., Oncotarget 9, (2018)
Waldhauer et al., mAbs 13, 1913791 (2021)
Gros et al., The Journal of Clinical Investigation 124, 2246-2259 (2014)
Gros et al., Nature Medicine 22, 433-438 (2016)
Codarri Deak et al., Nature 610, 161-172 (2022)
Ren et al., The Journal of Clinical Investigation 132, (2022)
Raeber et al., Science Translational Medicine 14, eabo5409 (2022)
Kalia et al., Immunity 32, 91-103 (2010)
Myszka et al., Protein Science 5, 2468-2478 (1996)
Davis et al., U.S. Pat. No. 10,556,952 (2016)
Skokos et al., Science Translational Medicine 12, eaaw7888 (2020)
van der Neut Kolfschoten et al., Science 317, 1554-1557 (2007)
Ward et al., The Journal of Immunology 201, 2579-2592 (2018)
Miragaia et al., Immunity 50, 493-504.e7 (2019)
Siddiqui et al., Immunity 50, 195-211.e10 (2019)
Miller et al., Nature Immunology 20, 326-336 (2019)
Fasso et al., Proceedings of the National Academy of Sciences 105, 3509-3514 (2008)
Teng et al., Cancer Research 75, 2139-2145 (2015)
Vilain et al., Clinical Cancer Research 23, 5024-5033 (2017)
Burova et al., Molecular Cancer Therapeutics 16, 861-870 (2017)
Crawford et al., Molecular Cancer Therapeutics 20, 1350-1358 (2021)
Arvedson et al., Annual Review of Cancer Biology 6, 17-34 (2022)
Belmontes et al., Science Translational Medicine 13, eabd1524 (2021)
Crawford et al., Science Translational Medicine 11, eaau7534 (2019)
Krieg et al., Proceedings of the National Academy of Sciences 107, 11906-11911 (2010)
Wu et al., Nature Cancer 4, 1309-1325 (2023)
LaPorte et al., Journal for ImmunoTherapy of Cancer 11, e006611 (2023)
Hashimoto et al., Nature 610, 173-181 (2022)
Lanitis et al., Annals of Oncology 28, xii18-xii32 (2017)
Tichet et al., Immunity 56, 162-179.e6 (2023)
Young et al., Cancer Discovery 12, 1625-1633 (2022)
Yan et al., J. Am. Soc. Mass Spectrom, 31:2171-2179 (2020)
Mosmann et al., The Journal of Immunology 138, 1813-1816 (1987)
Hofmann et al., International Immunopharmacology 6, 1902-1910 (2006)
Al-Haideri et al., Cancer Cell International 22, 365 (2022)
Macdonald et al., Proceedings of the National Academy of Sciences 111, 5147-5152 (2014)
Murphy et al., Proceedings of the National Academy of Sciences 111, 5153-5158 (2014)
Hao et al., Cell 184, 3573-3587.e29 (2021)
Kawalekar et al., Immunity 44, 380-90 (2016)

1. A method for treating or inhibiting the growth of a tumor, comprising:
(a) selecting a subject with cancer; and
(b) administering to the subject a first therapy comprising a therapeutically effective amount of a targeted immunocytokine in combination with a second therapy comprising a therapeutically effective amount of a programmed death 1 (PD1) inhibitor,
wherein administration of the combination leads to increased anti-tumor efficacy, as compared to a subject administered the targeted immunocytokine as monotherapy.

2. The method of claim 1, wherein the targeted immunocytokine comprises a fusion protein comprising (i) an immunoglobulin (Ig) antigen-binding domain that binds specifically to PD1 and (ii) an IL2 moiety.

3. (canceled)

4. (canceled)

5. The method of claim 2, wherein the IL2 moiety comprises: IL2 receptor alpha (IL2Ra) or a fragment thereof; and IL2 or a fragment thereof.

6. The method of claim 2, wherein the Ig antigen-binding domain of the checkpoint inhibitor comprises three heavy chain complementarity determining regions (CDRs) (HCDR1, HCDR2 and HCDR3) contained within a heavy chain variable region (HCVR) selected from the group consisting of SEQ ID NOs: 1, 11 and 20 and three light chain CDRs (LCDR1, LCDR2 and LCDR3) contained within a light chain variable region (LCVR) selected from the group consisting of SEQ ID NOs: 5 and 15.

7. The method of claim 2, wherein the Ig antigen-binding domain of the checkpoint inhibitor comprises three heavy chain complementarity determining regions (CDRs) (HCDR1, HCDR2, and HCDR3) and three light chain CDRs (LCDR1, LCDR2, and LCDR3), wherein:
HCDR1, HCDR2, HCDR3, LCDR1, LCDR2, and LCDR3 comprise the amino acid sequences of SEQ ID NOs: 2, 3, 4, 6, 7, and 8, respectively;
HCDR1, HCDR2, HCDR3, LCDR1, LCDR2, and LCDR3 comprise the amino acid sequences of SEQ ID NOs: 12, 13, 14, 16, 7, and 17, respectively; or
HCDR1, HCDR2, HCDR3, LCDR1, LCDR2, and LCDR3 comprise the amino acid sequences of SEQ ID NOs: 21, 22, 23, 6, 7, and 8, respectively.

8. The method of claim 2, wherein the Ig antigen-binding domain of the checkpoint inhibitor comprises a HCVR/LCVR amino acid sequence pair selected from SEQ ID NOs: 1/5, 11/15, and 20/5.

9. The method of claim 2, wherein the Ig antigen-binding domain of the checkpoint inhibitor comprises a HCVR/LCVR amino acid sequence pair of SEQ ID NOs: 20/5.

10. The method of claim 2, wherein the fusion protein comprises a heavy chain comprising a HCVR and a heavy chain constant region of IgG1 or IgG4 isotype.

11. The method of claim 2, wherein the fusion protein comprises a heavy chain and a light chain, wherein:
the heavy chain comprises the amino acid sequence of SEQ ID NO: 24, and the light chain comprises the amino acid sequence of SEQ ID NO: 25;
the heavy chain comprises the amino acid sequence of SEQ ID NO: 9, and the light chain comprises the amino acid sequence of SEQ ID NO: 10; or
the heavy chain comprises the amino acid sequence of SEQ ID NO: 18, and the light chain comprises the amino acid sequence of SEQ ID NO: 19.

12. The method of claim 10, wherein the IL2 moiety is attached to the C-terminus of the heavy chain via a linker comprising the amino acid sequence of SEQ ID NO: 30 or 31.

13. The method of claim 2, wherein the IL2 moiety comprises IL2 having the amino acid sequence of SEQ ID NO: 29.

14. The method of claim 2, wherein the IL2 moiety comprises the IL2 or fragment thereof connected via a linker to the C-terminus of the IL2Ra or fragment thereof.

15. The method of claim 14, wherein the IL2Ra or fragment thereof comprises the amino acid sequence of SEQ ID NO: 28.

16. The method of claim 2, wherein the IL2 moiety comprises the amino acid sequence of SEQ ID NO: 27.

17. The method of claim 1, wherein the PD1 inhibitor of the second therapy comprises an antibody or antigen-binding fragment thereof that binds specifically to PD1, PD-L1 or PD-L2, or a bioequivalent thereof.

18. The method of claim 1, wherein the PD1 inhibitor of the second therapy is an anti-PD1 antibody selected from cemiplimab, nivolumab, pembrolizumab, pidilizumab, MEDI0608, BI 754048, PF-06371548, spartalizumab, camrelizumab, JNJ-63313240, and MCLA-134.

19. The method of claim 1, wherein the PD1 inhibitor of the second therapy is an anti-PD-L1 antibody selected from REGN3504, avelumab, atezolizumab, durvalumab, MDX-1105, LY3300054, FAZ053, STI-1014, CX-031, KN035, and CK-301.

20. The method of claim 1, wherein the PD1 inhibitor of the second therapy is an antibody or antigen-binding fragment thereof that binds specifically to PD1 and comprises three heavy chain complementarity determining regions (CDRs) (HCDR1, HCDR2 and HCDR3) contained within a heavy chain variable region (HCVR) of SEQ ID NO: 33 and three light chain CDRs (LCDR1, LCDR2 and LCDR3) contained within a light chain variable region (LCVR) of SEQ ID NO: 34.

21. The method of claim 20, wherein the HCDR1, HCDR2, HCDR3, LCDR1, LCDR2, and LCDR3 of the anti-PD1 antibody or antigen-binding fragment thereof of the second therapy comprise the amino acid sequences of SEQ ID NOs: 35, 36, 37, 38, 39, and 40, respectively.

22. The method of claim 21, wherein the antibody or antigen-binding fragment thereof of the second therapy that binds specifically to PD1 comprises a HCVR comprising the amino acid sequence of SEQ ID NO: 33 and/or a LCVR comprising the amino acid sequence of SEQ ID NO: 34.

23. The method of claim 20, wherein the antibody of the second therapy that binds specifically to PD1 comprises a heavy chain and a light chain, wherein the heavy chain has an amino acid sequence of SEQ ID NO: 41 and/or the light chain has an amino acid sequence of SEQ ID NO: 42.

24. The method of claim 17, wherein the PD1 inhibitor of the second therapy comprises is cemiplimab or a bioequivalent thereof.

25. The method of claim 1, wherein the cancer is selected from anal cancer, angiosarcoma, basal cell carcinoma, bladder cancer, bone cancer, brain cancer, breast cancer, cervical cancer, cholangiocarcinoma, chondrosarcoma, colon cancer, colorectal cancer, cutaneous squamous cell carcinoma, endometrial cancer, esophageal cancer, glioblastoma multiforme, head and neck squamous cell cancer, hepatocellular carcinoma, kidney cancer, liver cancer, lung cancer, Merkel cell carcinoma, melanoma, myeloma, non-small cell lung cancer, ovarian cancer, pancreatic cancer, prostate cancer, salivary gland cancer, skin cancer, soft tissue sarcoma, stomach cancer, testicular cancer, and uterine cancer.

26. The method of claim 1, wherein the targeted immunocytokine is administered to the subject before or concurrently with the administration of the PD1 inhibitor.

27. The method of claim 1, wherein the targeted immunocytokine is administered to the subject after the administration of the PD1 inhibitor.

28. A method for increasing the efficacy of targeted immunocytokine therapy, comprising:
(a) selecting a subject with cancer; and
(b) administering to the subject a first therapy comprising a therapeutically effective amount of a targeted immunocytokine in combination with a second therapy comprising a therapeutically effective amount of a programmed death 1 (PD1) inhibitor,
wherein administration of the combination leads to increased anti-tumor efficacy, as compared to a subject administered the targeted immunocytokine as monotherapy.

29. The method of claim 28, wherein the targeted immunocytokine comprises a fusion protein comprising (a) an immunoglobulin antigen-binding domain comprising a heavy chain variable region having an amino acid sequence of SEQ ID NO: 20 and a light chain variable region having an amino acid sequence of SEQ ID NO: 5; and (b) an IL2 moiety comprising IL2 or a fragment thereof connected via a linker to the C-terminus of IL2Ra or fragment thereof.

30. The method of claim 28, wherein the PD1 inhibitor of the second therapy is an antibody or antigen-binding fragment thereof that binds specifically to PD1 and comprises three heavy chain complementarity determining regions (CDRs) (HCDR1, HCDR2 and HCDR3) contained within a heavy chain variable region (HCVR) of SEQ ID NO: 33 and three light chain CDRs (LCDR1, LCDR2 and LCDR3) contained within a light chain variable region (LCVR) of SEQ ID NO: 34.
